(19) United States
(12) Patent Application Publication
(10) Pub. No.: US 2006/0154236 A1
(43) Pub. Date: Jul. 13, 2006

(54) Computer-assisted analysis

(76) Inventors: Steven J. Altschuler, Dallas, TX (US); Lani Wu, Dallas, TX (US); Michael D. Slack, Dallas, TX (US)

Correspondence Address: CHOATE, HALL & STEWART LLP, TWO INTERNATIONAL PLACE, BOSTON, MA, 02110, US

(21) Appl. No.: 11/271,215

(22) Filed: Nov. 11, 2005

Related U.S. Application Data
(60) Provisional application No. 60/626,892, filed on Nov. 11, 2004.

(51) Int. Cl. C12Q 1/00; G06F 19/00

(52) U.S. Cl. 435/4; 702/19

(57) ABSTRACT

The present invention provides methods and systems for automated morphological analysis of cells also known as phenotypic screening. The inventive methods are particularly useful in the rapid analysis of cells required in a biological screen or in the screening for agents with a particular mechanism of action. Agents which cause a particular phenotype in the cells can be identified using the inventive quantitative morphometric analysis of cells. The data gathered using the inventive method can also be quantified and analyzed later for various trends and classifications (e.g., Kolmogorov-Smirnov statistics, titration-invariant similarity scores). Characteristics of cells which can be determined using this method include number of nuclei, size of cell, size of nuclei, number of the centrosomes, shape of cells, size of centrosomes, perimeter of nucleus, shape of nucleus, staining for a particular protein, staining for an organelle, pattern of staining, and degree of staining.

### RELATED APPLICATIONS

The present application claims priority under 35 U.S.C. § 119(e) to U.S. provisional application, U.S. Ser. No. 60/626,892, entitled “Computer-Assisted Cell Analysis,” filed Nov. 11, 2004, the entire contents of which is incorporated herein by reference. The present application is also related to U.S. application, U.S. Ser. No. 10/425,827, entitled “Computer-Assisted Cell Analysis”, filed May 12, 2003, and U.S. provisional application, U.S. Ser. No. 60/379,296, entitled “Computer-Assisted Cell Analysis”, filed May 10, 2002, the entire contents of each of which are incorporated herein by reference.

### Government Support

The work described herein was supported, in part, by grants from the National Institutes of Health (GM062566 and CA078048). The United States government may have certain rights in the invention.

### BACKGROUND OF THE INVENTION

The impetus to design better screens for identifying chemical compounds with a desired biological activity has been heightened over the past decade with the advent of combinatorial chemistry. Organic chemists are now able to produce thousands to millions of compounds in parallel while achieving a high degree of chemical diversity. These new compounds are subsequently assayed or screened to identify compounds with a particular activity. Typically, a library of compounds is put through one assay at a time to look for a particular activity with most of the compounds not having the desired activity being assayed for.

Many of these screens and assays include exposing cells to a chemical compound and observing the effect of the compound on the cell. The exposure to the chemical compound may lead to inhibition of growth, to proliferation, to cell death, etc. resulting in the determination of concentrations at which 50% growth inhibition occurs, total growth inhibition occurs, and 50% lethality occurs, for example. However, the determination of these few data points for a particular compound at a particular concentration is labor intensive and much data is lost by focusing on just certain aspects of the cells being cultured and exposed to the chemical compound.

High-throughput techniques for describing cell phenotype such as transcriptional and proteomic profiling allow, quantitative and machine readable measures of the response of cell populations to perturbation (Eisen et al. Proc. Natl. Acad. Sci. USA 95:14863-68, 1998; Gavin et al. Nature 415:141-47, 2002; Yo et al. Nature 415:180-83, 2002; Uetz et al. Nature 403:623-27, 2000; each of which is incorporated herein by reference). However, although transcriptional and proteomic profiling are powerful in analyzing the transcription of a variety of genes and levels of proteins, respectively, they only look at the levels of transcription of genes and at protein levels, and not at cells as a whole (i.e., the cell's phenotype). Automated microscopy has the potential to complement these profiling approaches, by allowing fast, cheap data collection that offers a wealth of information about protein behaviors within individual cells that can be directly related to biological pathways (Murphy et al. Proc. Int. Conf Intell. Syst. Mol. Biol. 8:251-59, 2000; Price et al. J. Cell Biochem. Suppl. 39:194-210, 2002; each of which is incorporated herein by reference).

Accessing these data and using them to produce useful profiles of cell phenotype will require new methods of automated image analysis, which have so far lagged behind the adoption of high-throughput imaging technologies.

### SUMMARY OF THE INVENTION

The present invention stems from the recognition that many biological screens, which use cytological analysis, in drug development, pathology, cell biology, and genomics require the microscopic analysis of cell samples. This work is usually carried out by a trained human microscope operator who laboriously looks at plates or wells of cells to find the cells with the desired phenotype. Because this type of work requires a trained human operator, it is very costly and time-consuming, and it is subject to human error especially when the operator becomes fatigued after looking at many samples. Also, with a human operator the results are not readily quantifiable and are usually limited to a handful of easily observable characteristics of the cells, and the data analysis may be limited to a scoring system designed for a particular experiment at the very beginning of the experiment. If later different aspects of the cells are to be analyzed or a different scoring system is to be used, the work must be repeated from the beginning.

The present invention provides methods and systems for automating the analysis of cells. The methods termed phenotypic screening can be used to describe the physiological state of cells based on the automated collection of data from image processing software and statistical analysis of this data. One of the advantages of this method is that the data is broad, computable, and different than the data collected from transcriptional profiling or proteomic profiling experiments. In certain embodiments, the inventive method is a phenotype-based screening method for quantitative morphometric analysis of cells used to describe and quantitate the mechanism and specificity of drugs or drug candidates. An image of the cells is analyzed by a computer running image processing software designed to determine the various states, morphologies, appearances, characteristics, staining patterns, and/or conditions of the cells in the image. The aspects of the cells in the image to be analyzed include number of cells in the image, pixel area of each cell, perimeter of each cell, volume of each cell, ellipticity of each cell, shape of each cell, number of nuclei per cell, pixel area of each nucleus, perimeter of each nucleus, volume of each nucleus, shape of each nucleus, pixel area of nucleus, degree of staining for nucleic acid in each nucleus, number of centromeres per cell, average cross-sectional area of cells, morphology, eccentricity, degree of staining for a cytoplasmic protein, degree of staining for a nuclear protein, degree of staining for an organelle, pattern of staining, etc. These aspects of a cell or cell population may be quantified and used to determine the physiological or biochemical status of the cells imaged (e.g., what phase of the cell cycle the cells are in, whether the cells are starved, whether the cells are dividing, whether the cells are dieing, whether the cells are differentiating, whether the cells are undergoing apoptosis, whether protein synthesis has been inhibited, whether DNA synthesis has been inhibited, whether transcription has been inhibited). In certain embodiments, the cells are not labeled or modified before imaging, and in other embodiments, the cells may be fixed and/or labeled for various cellular organelles, nucleic acids such as DNA and RNA, protein, specific proteins (e.g., p53, cFos, p38, pERK, etc.), etc. Any type of cells may be used in the present invention (e.g., cells derived from laboratory cell lines, cells from a biopsy, cells derived from any species, bacterial cells, human cells, yeast cells, mammalian cells, etc.) In certain embodiments, the genomes of the cells have not been altered. In other embodiments, the genomes of the cells have been altered.

In one aspect, the Kolmogorov-Smirnov non-parametric statistic is calculated for a particular aspect(s) of the cells (also known as a descriptor) in a single image. The Kolnogorov-Smirnov statistic (K-S statistic) is useful because a single image may contain cells in many different states. Therefore, measurements of certain aspects of a cell may produce distributions that are difficult to reduce to simple parametric models. The K-S statistic is calculated from the continuous distribution function for a descriptor. The K-S statistic is defined as the difference between two continuous distribution functions (e.g., treated versus untreated) at the point where the difference between the functions reaches a maximum (i.e., the function KS(f,g) computes f−g at the point where |f−g| reaches its maximum. The K-S statistic may be normalized by dividing it by a measure of the variability of the descriptor within a population such as a control population. To better visualize these scores, this normalized score can then be displayed in a heat plot by assigning the score to a color.

In another aspect, the effect of an agent on a cell is complex, and profiling is performed as a function of drug concentration since the effect of a drug is typically dose-dependent. These complex effects may be due to differential sensitivity of downstream pathways to degree of perturbation of a primary target, or binding of drugs to multiple targets with different affinities, for example. In certain embodiments, a titration-invariant similarity score (TISS) is calculated for analyzing dose-dependent responses. The TISS is particularly useful in assessing the similarity or dissimilarity of test compounds independent of the starting point of the titration series. For example, in determining drug mechanisms changes in specificity are relevant, but changes in affinity (e.g., primary effective concentration) are not. A TISS was developed to allow comparison between dose-response profiles independent of starting dose. TISS values may be particularly useful in clustering to group test compounds with similar mechanisms of action. In certain embodiments, the TISS between two compounds is calculated as follows: (a) first a titration sub-series for each compound to account for different possible starting concentrations is defined; (b) a correlation for pairs of these sub-series is defined; and (c) a similarity measure derived from the strongest correlation over a determined range of these sub-series is defined. Descriptor vectors may also be compared using the above analysis.

In certain aspects, the computer analysis of cell samples is used in biological screens where hundred to thousands of cell samples are to be analyzed. This analysis is particularly useful in analyzing arrays of cells in which the cells in each well or plate have been treated with a particular agent (e.g., drugs, chemical compounds, small molecules, peptides, proteins, biological molecules, polynucleotides, anti-sense agents). The method is particularly useful in the field of high throughput screening. By analyzing the cells for various characteristics such as morphology, number of nuclei, number of centromeres, cell shape, volume of cell, volume of nuclei, etc. using a computer running the visual analysis software, one can screen a vast number of agents over a range of titrations fairly quickly to identify those with a particular biological activity. For example, using this method one could identify agents that would be useful as anti-neoplastic agents by searching for agents that decrease the number of cells in the microscopic field, decrease the number of nuclei, and/or decrease the number of centromeres, that is searching for a microscopic field of cells that are not undergoing mitosis. In another example, one may screen known compounds such as an antibiotic (e.g., penicillin) to look for its effect on various visual characteristics of treated cells. Once these effects are known, one could then look for agents with a similar morphological effect on cells. In this manner, one could quickly screen for novel agents with effects similar to those of known pharmacological agents. In certain embodiments, agents for which the mechanism of action is not known are analyzed using the inventive system and compared to reference data collected from compounds with known mechanisms of action to determine the mechanism of action of the test agent. In certain embodiments, this analysis is performed using clustering algorithms.

The invention also provides a system for carrying out the inventive methods. The system may include a microscope able to acquire images at various magnifications or resolutions, a microprocessor, and software for carrying out the image analysis and the statistical analysis of the raw data derived from the images. In certain embodiments, the system includes the hardware and/or software necessary to calculate titration-invariant similarity scores (TISSs). I other embodiments, the system includes the hardware and/or software necessary to perform clustering analysis. In certain embodiments, a low magnification is useful where many cells are to be analyzed. In other embodiments, a high magnification is useful when analyzing for a characteristic only visible at high power. In addition to magnification, the resolution of the image may be varied depending on the analysis to be performed. In certain embodiments, a low resolution image is preferred for carrying out the automated analysis. The system may also include a storage device for storing the images and/or data for future recall if need be.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1 shows two views of phenotype-transcriptional profiling and cytological profiling.

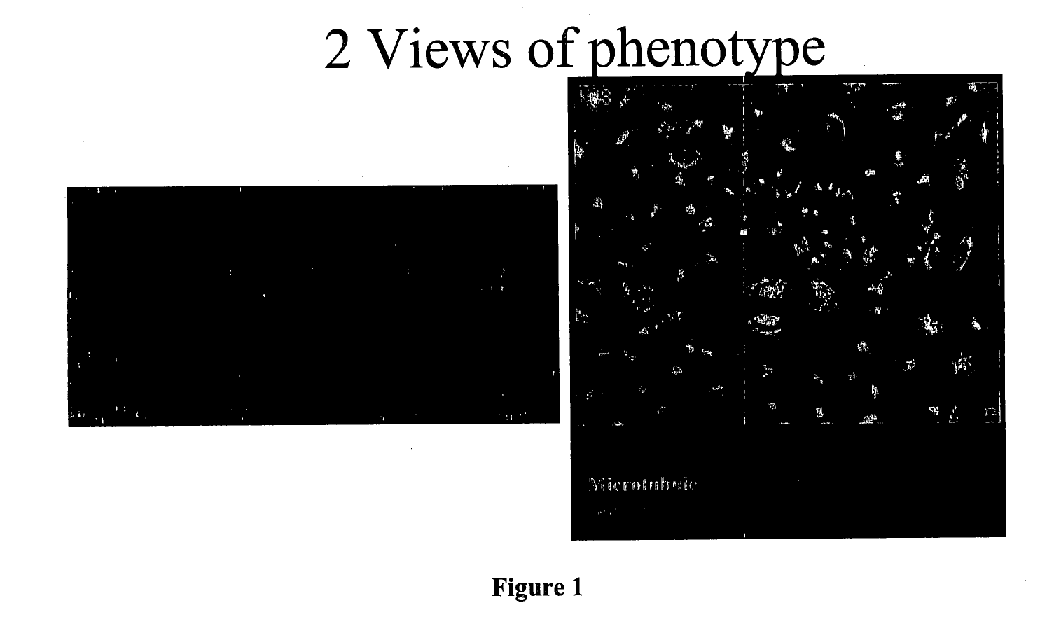

FIG. 2 shows a diagram of how cytological profiling can be used in high throughput analysis.

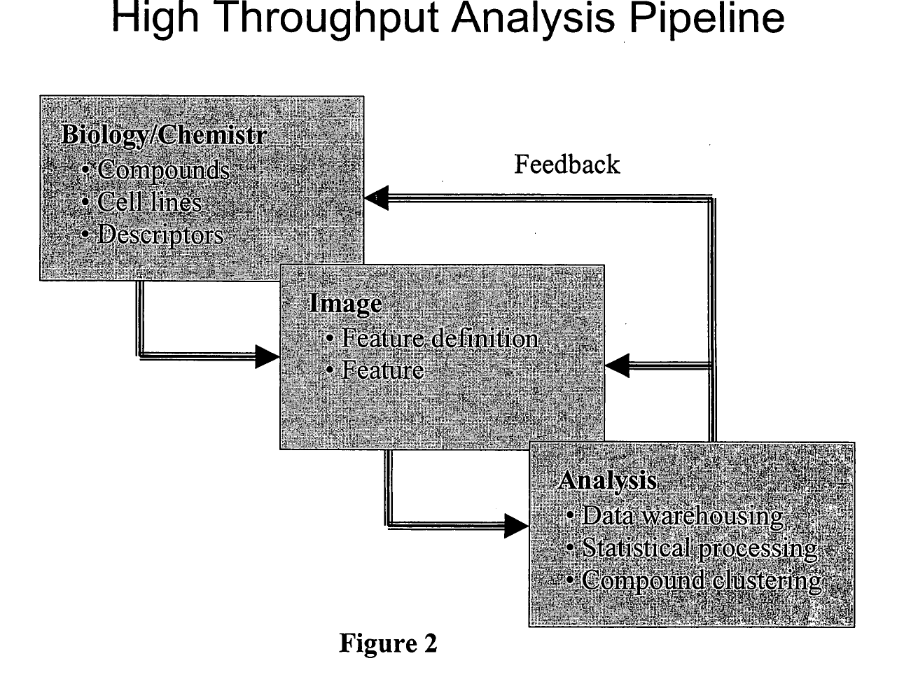

FIG. 3 shows the design of a typical experiment involving 80 compounds at various concentrations to yield over 10 million measurements or 6 GB of numerical data.

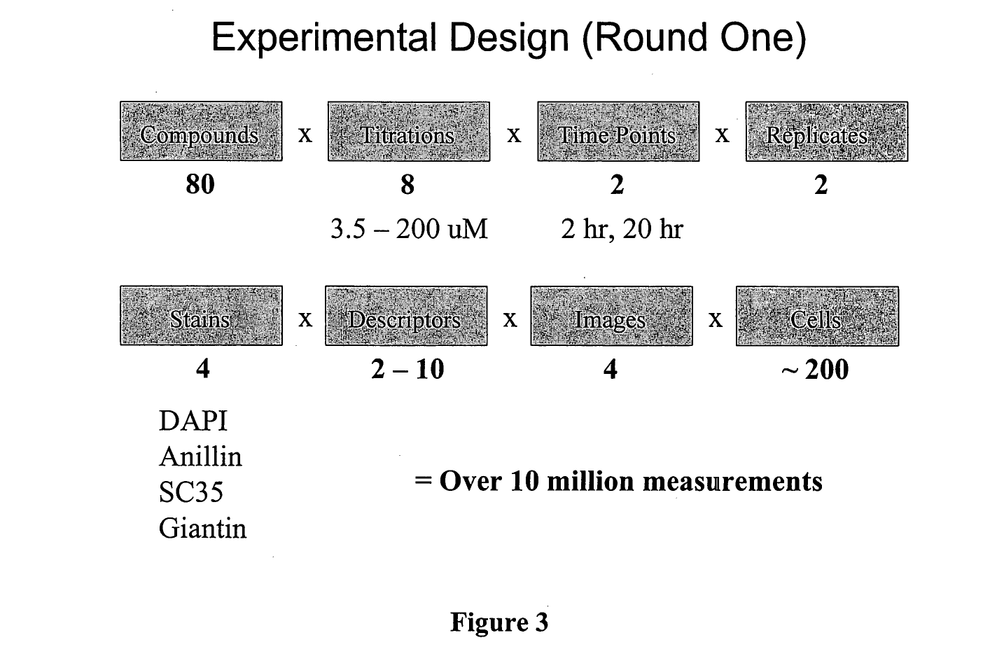

FIG. 4 shows the imaging of cells, processing of the image, measurement of shape and intensity values for each object, and statistical analysis.

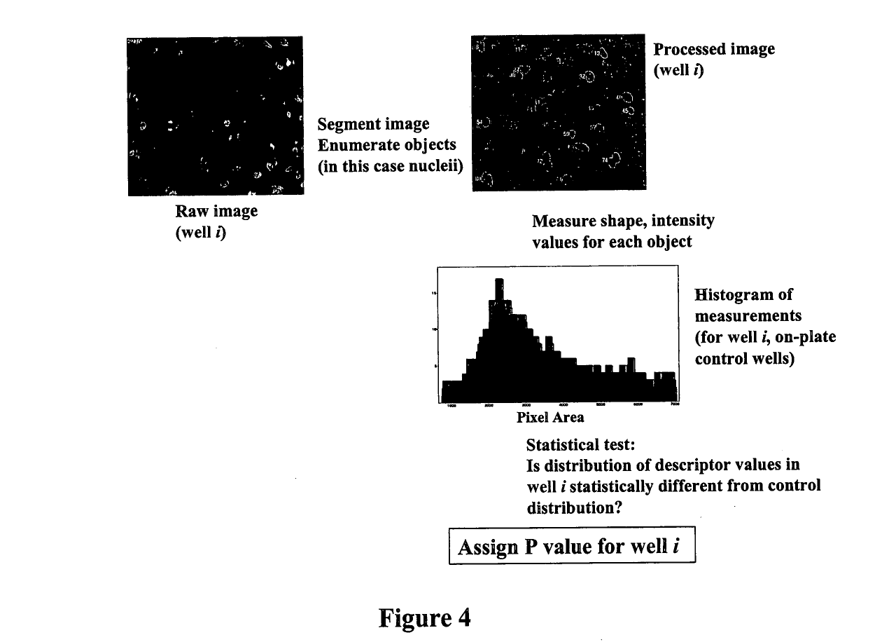

FIG. 5 shows the nine descriptors used in the experiment outlined in FIG. 3.

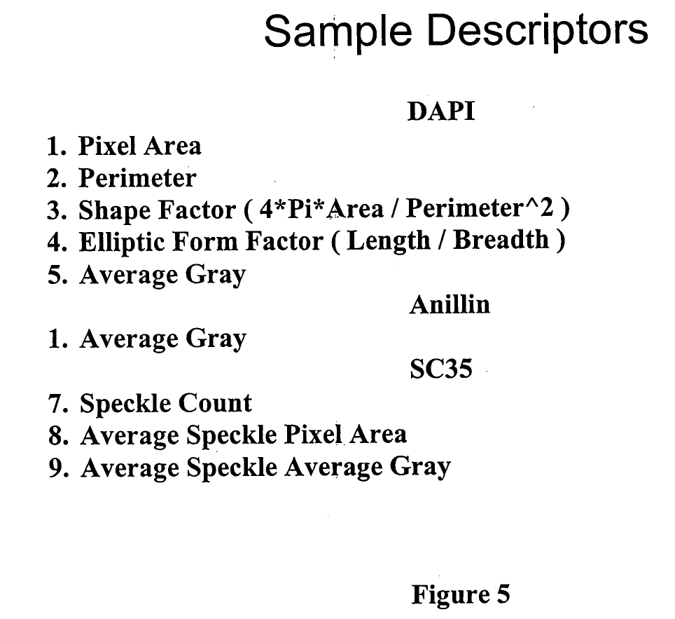

FIG. 6 shows two distributions of the average gray descriptor using the DAPI stain with cells contacted with cytochalasin D.

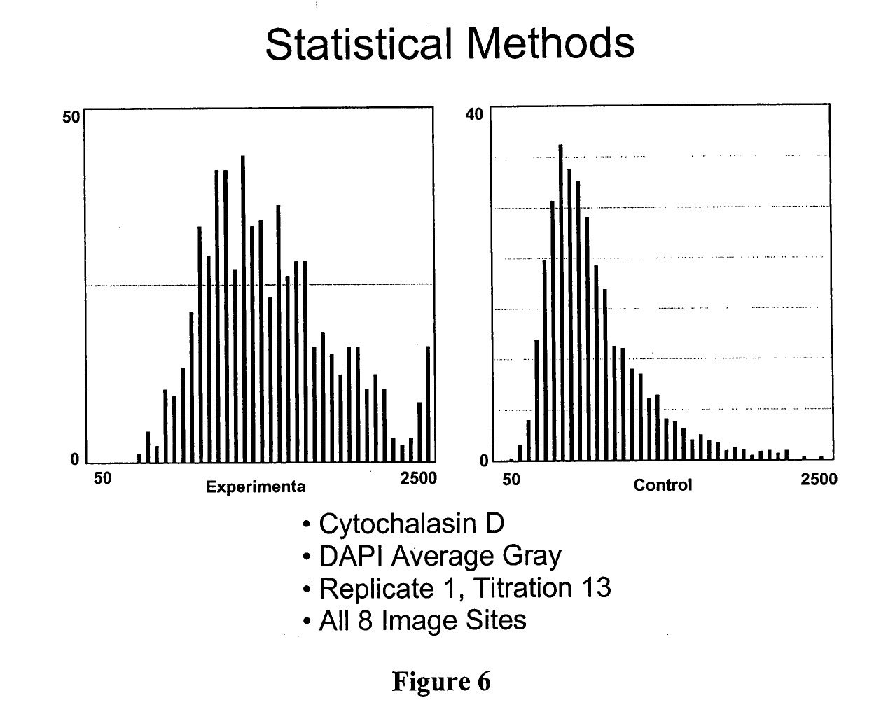

FIG. 7 shows a KS plot of the DAPI pixel area (nuclear size) descriptor at 20 hours for 40 compounds at different dilutions and an untreated control.

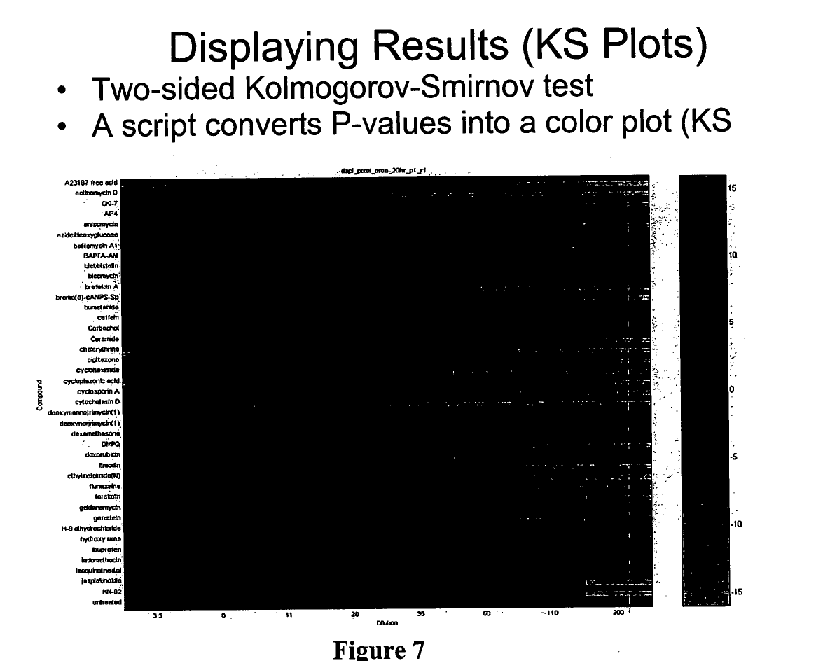

FIG. 8 shows the expanded KS plot of the nuclear size descriptor at 20 hours for actinomycin D, blebbistatin, brefeldin A, cycloheximide, and doxorubicin at eight different concentrations.

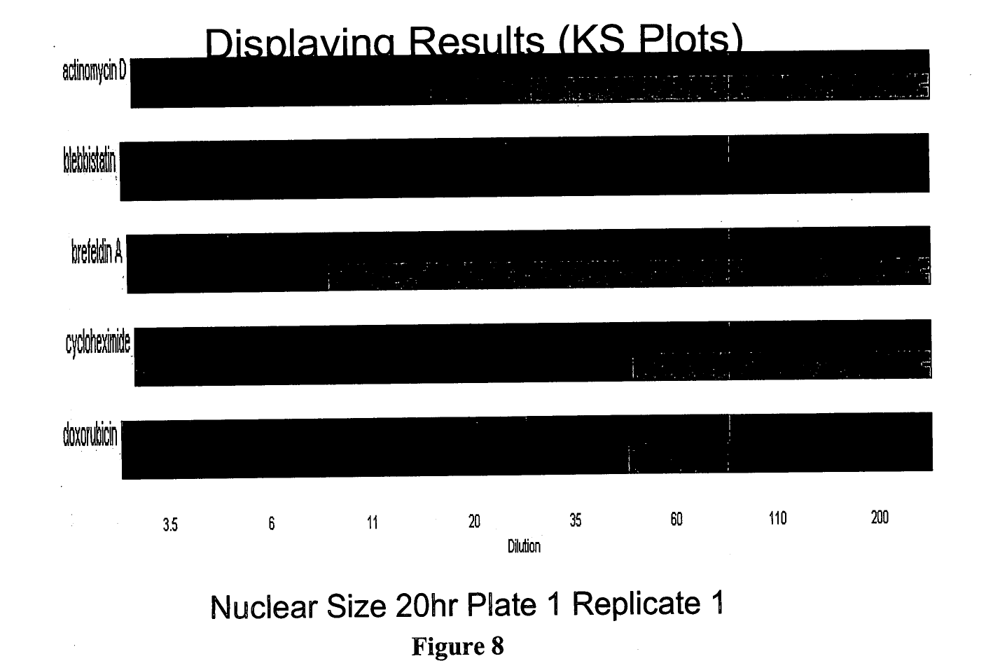

FIG. 9 shows the interpretation of the KS plots.

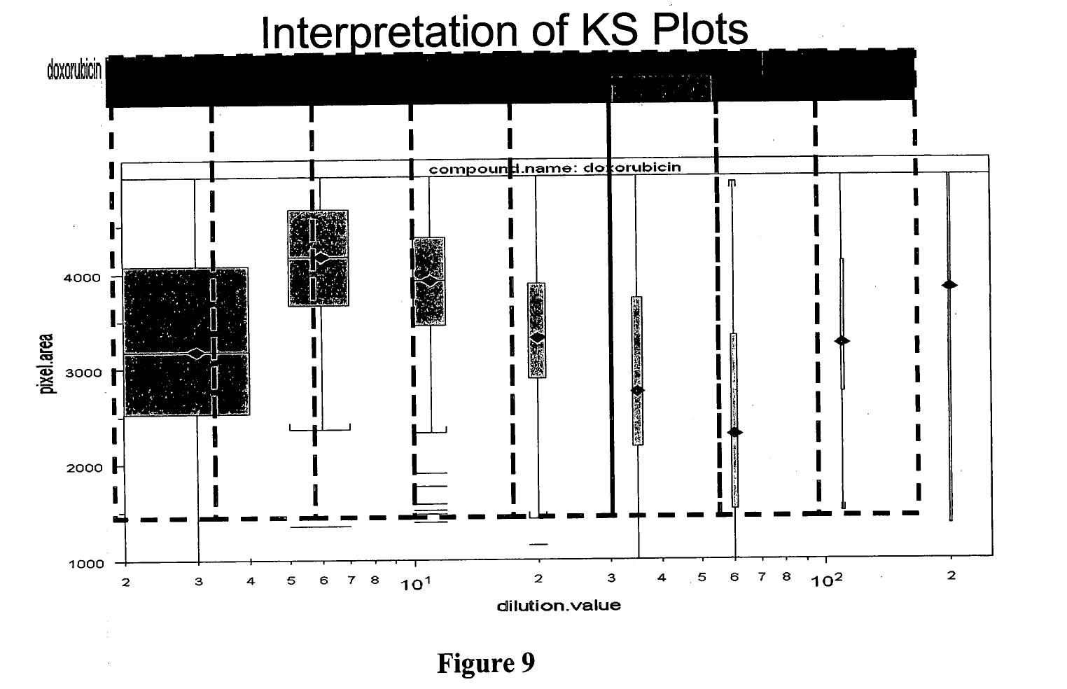

FIG. 10 shows the KS plot for nuclear size for brefeldin A, dexamethasone, doxorubicin, and control, and the corresponding images.

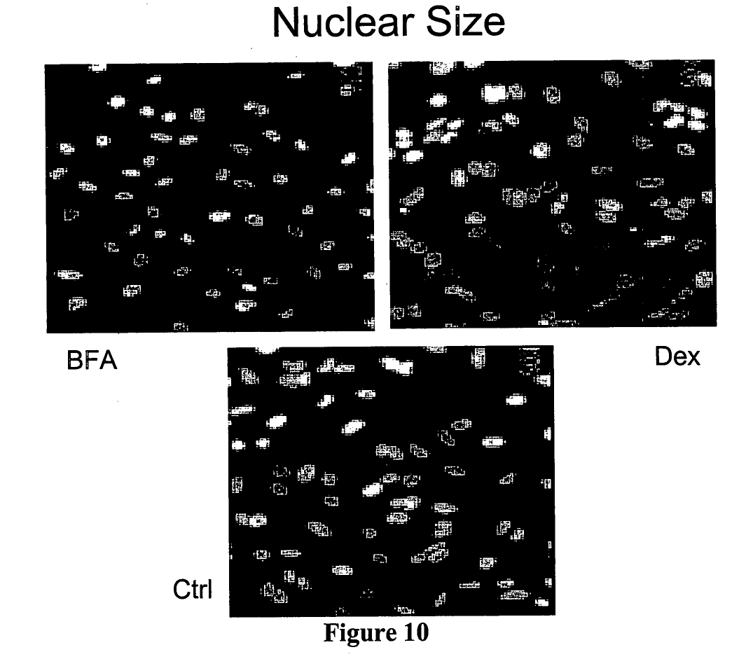

FIG. 11 shows the KS plot for nuclear speckle count for actinomycin D, brefeldin A, doxorubicin, and untreated control, and corresponding images.

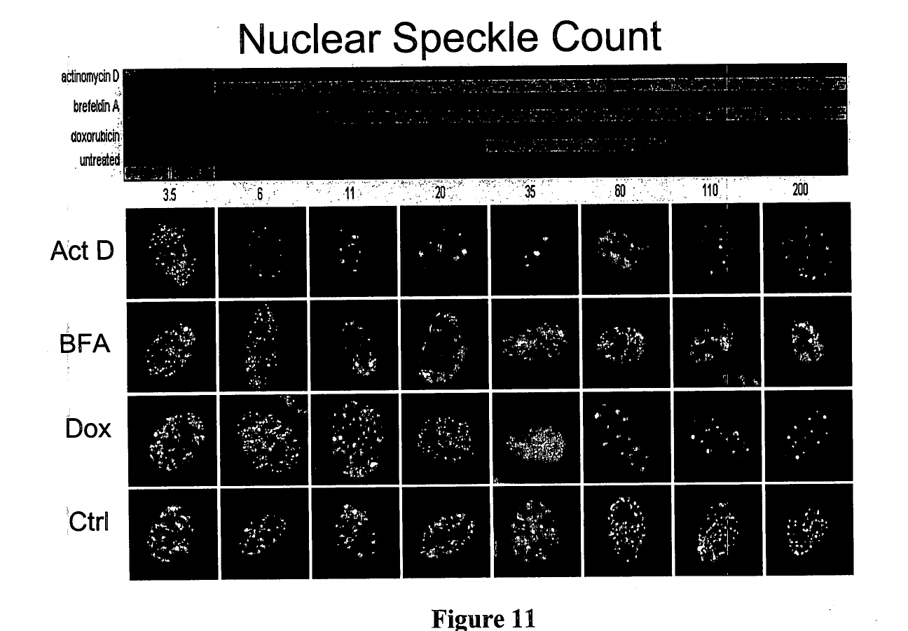

FIG. 12 shows the empirical cumulative distribution function of the control and experimental distributions and the calculation of the Kolmogorov-Smirnov statistic.

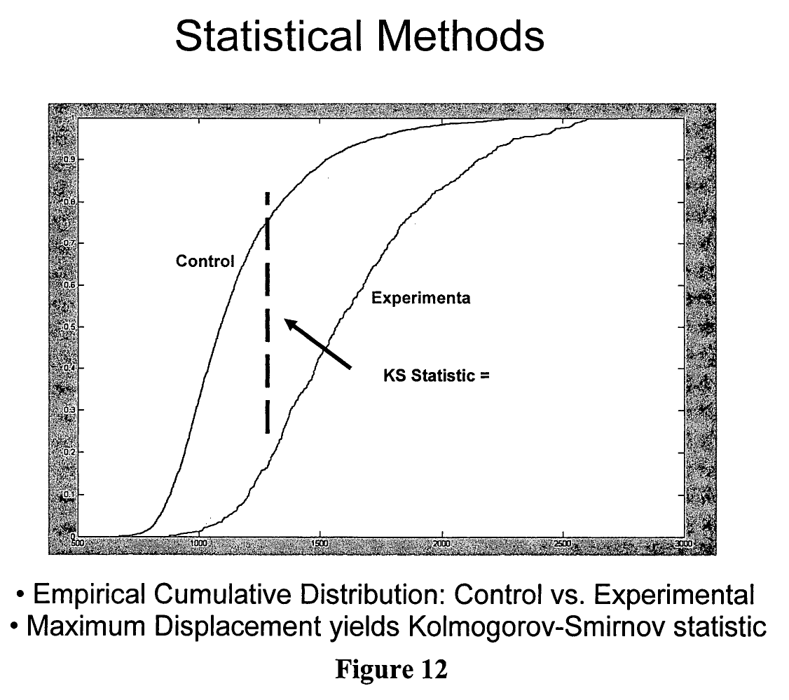

FIG. 13 displays the results using a KS plot of the nine descriptor (two replicates) for cytochalasin D.

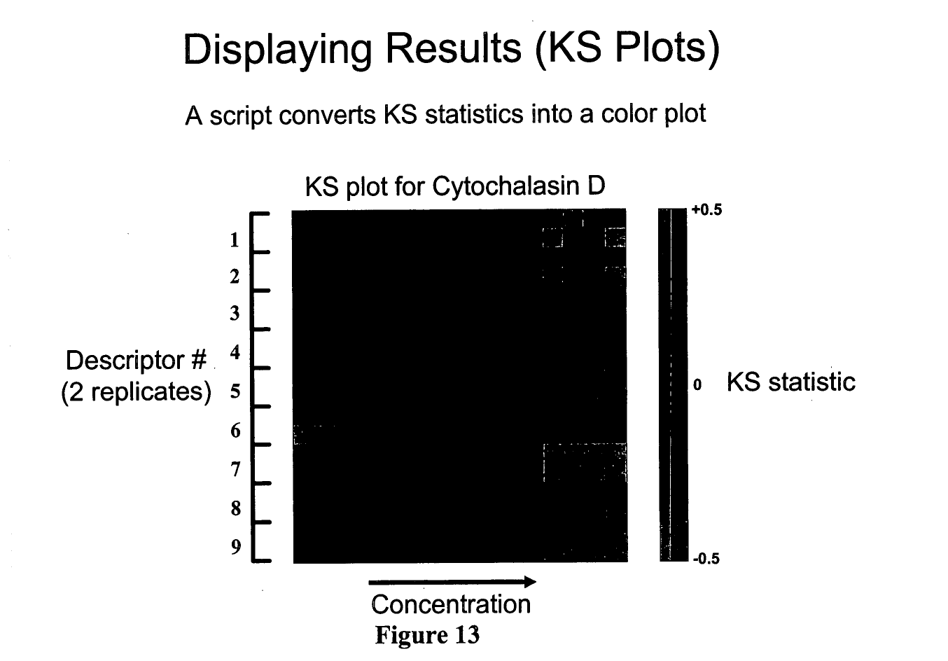

FIG. 14 is a KS plot showing a noisy descriptor and replicates that do not seem to be very reproducible.

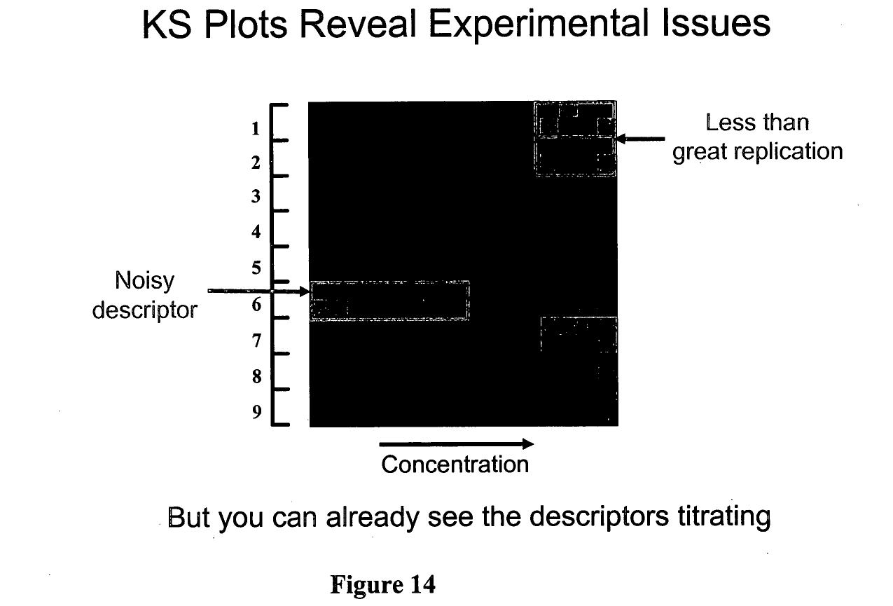

FIG. 15 shows the KS data for three compounds, cytochalasin D, jasplakinoldie, and latrunculin B, which are known to affect actin metabolism.

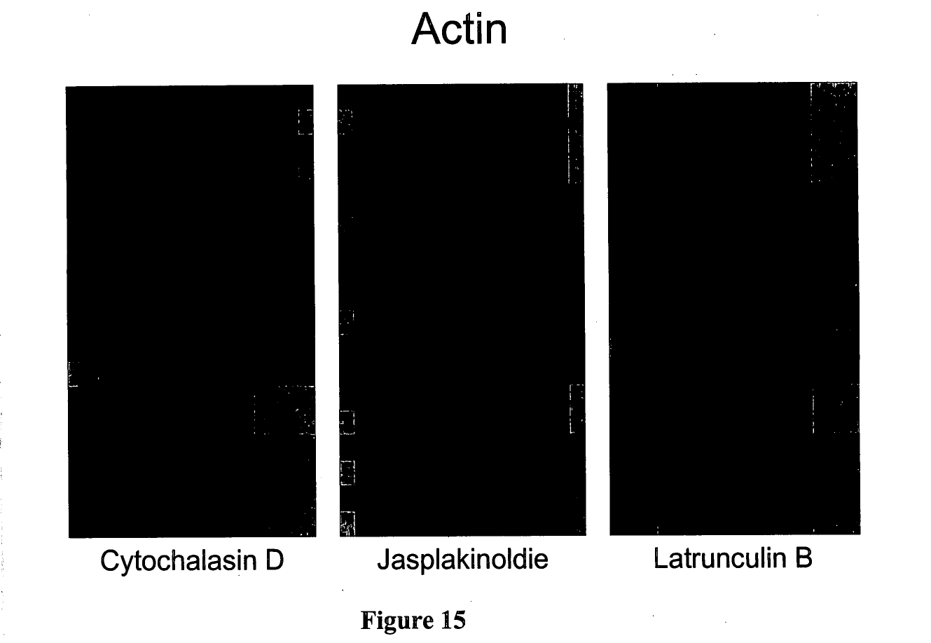

FIG. 16 shows the KS data for three compounds, 105D, colchicine, and griseofulvin, which are known to affect tubulin metabolism.

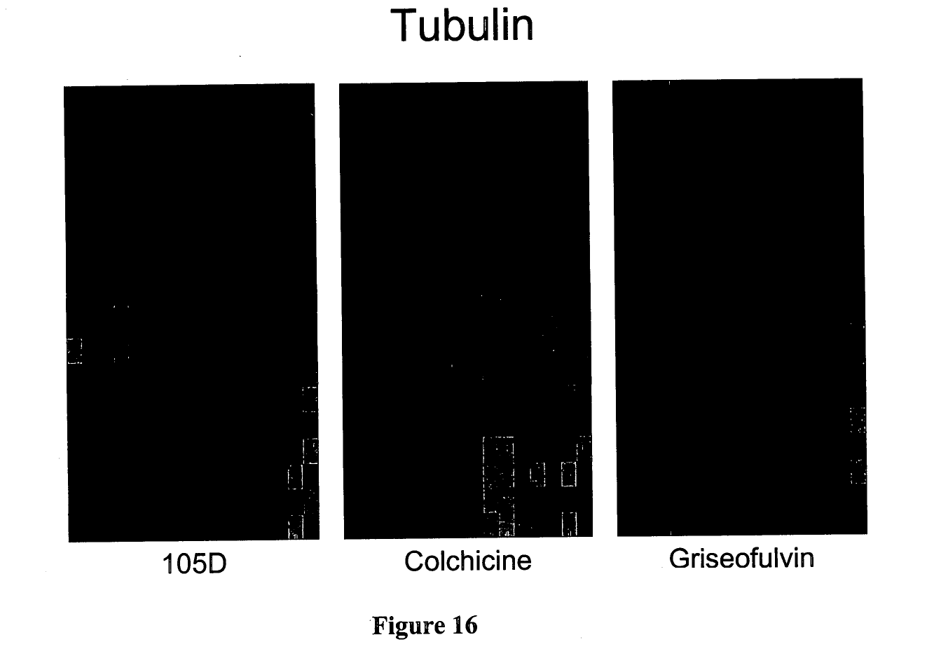

FIG. 17 shows the KS data for three compounds, nocodazole, podophyllotoxin, and taxol, which are known to affect tubulin metabolism.

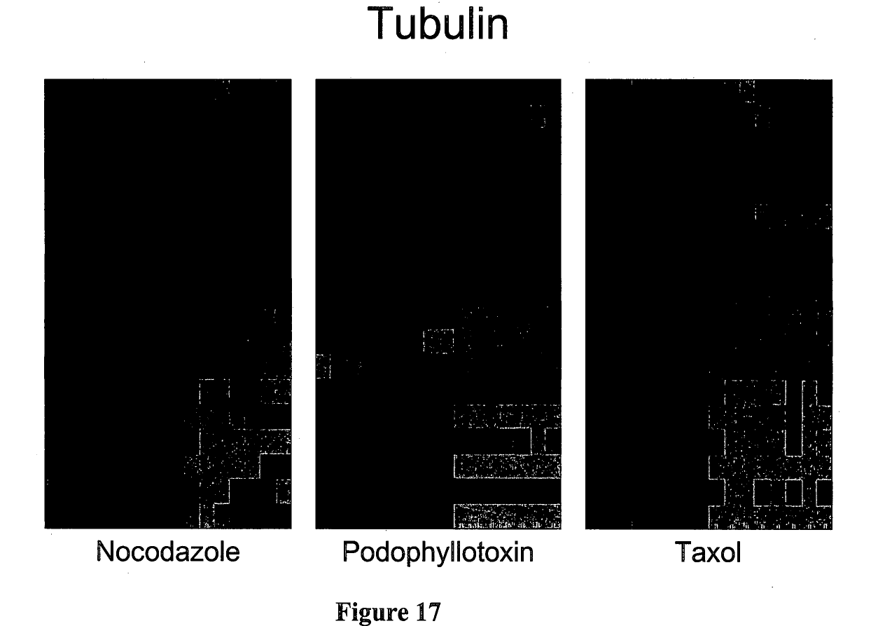

FIG. 18 shows the KS data for vinblastine, which is known to affect tubulin metabolism.

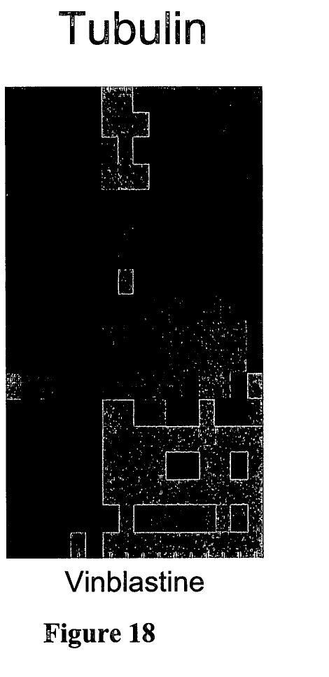

FIG. 19 shows the KS data for camptothecin, doxorubicin, and etoposide, which are known to affect topoisomerase activity.

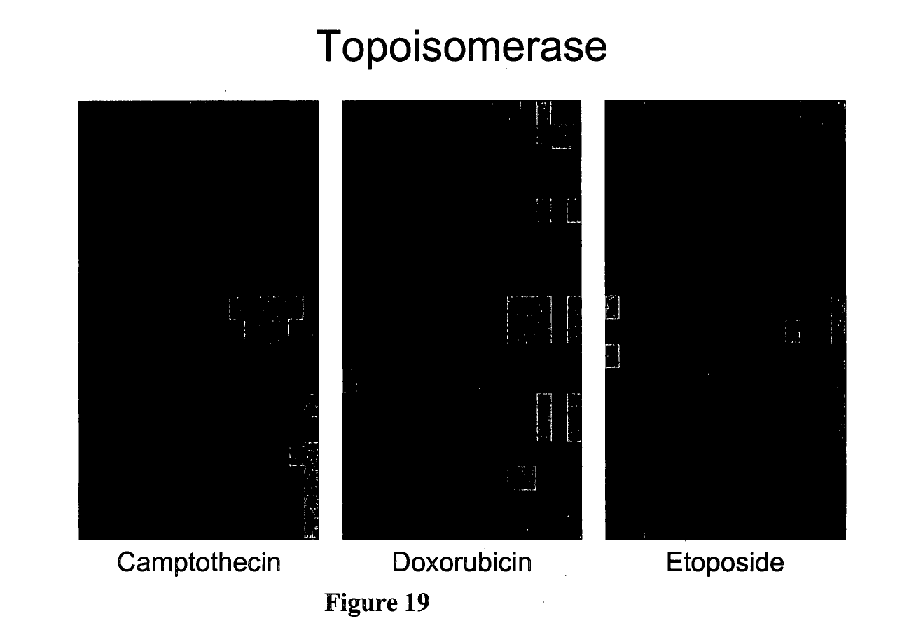

FIG. 20 shows the KS data from anisomycin, cycloheximide, and emetine, which are known to bind to ribosome and affect protein synthesis in cells.

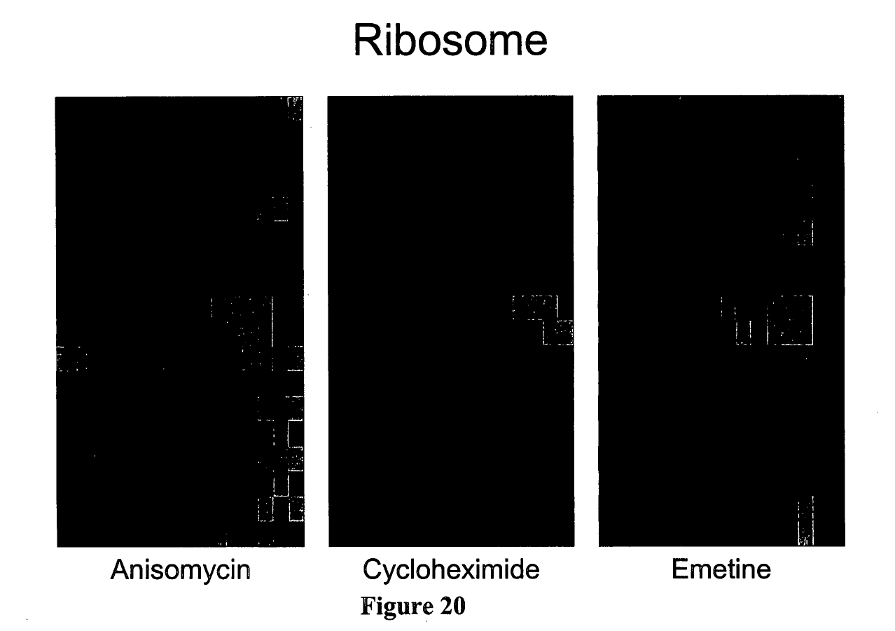

FIG. 21 shows the KS data from puromycin, which is also known to bind ribosomes and thereby affect protein synthesis in cells.

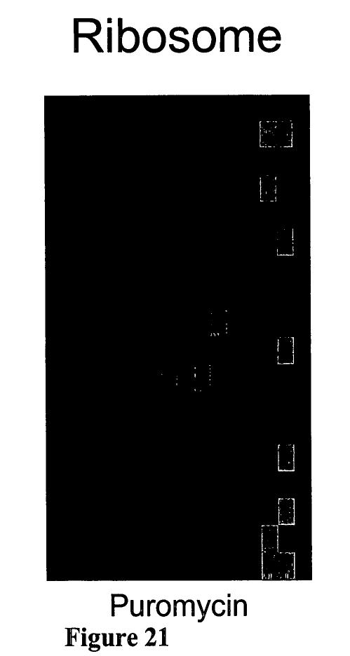

FIG. 22 shows the KS data from ibuprofen, indomethacin, and sulindac sulfide, which are inhibitors of cyclooxygenase.

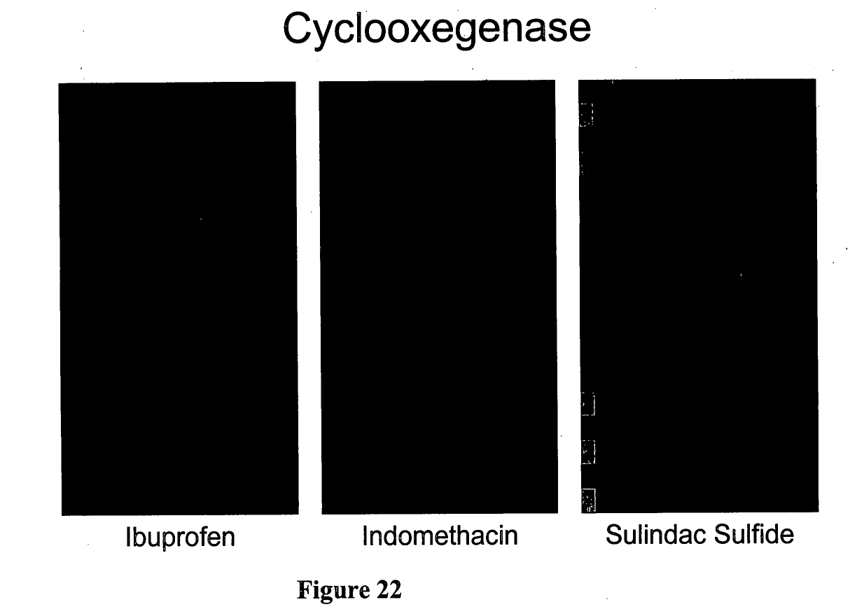

FIG. 23 shows the KS data from alsterpaullone, indirubin monoxime, and olomucine, which inhibits CDK.

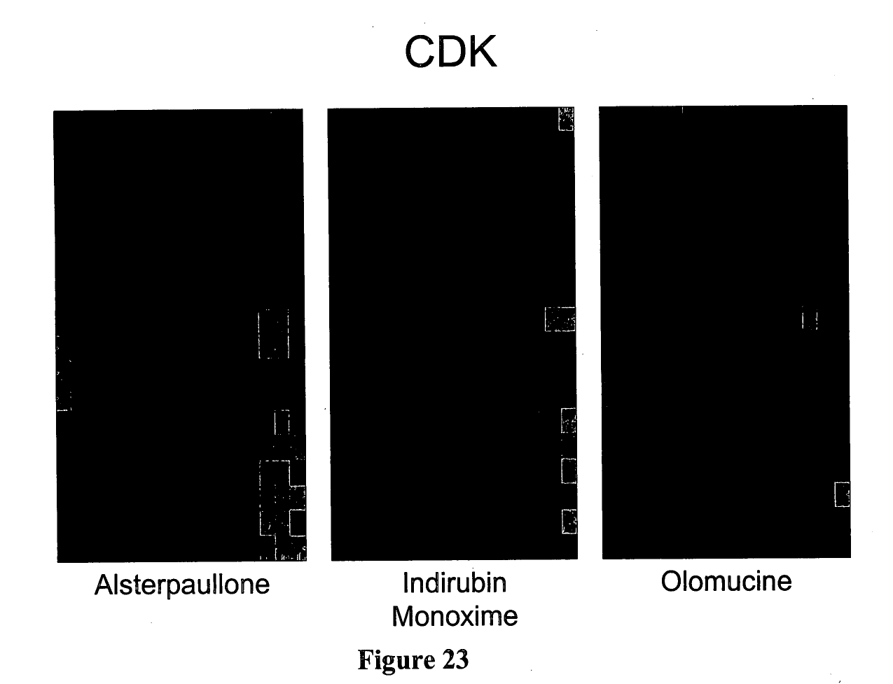

FIG. 24 shows the KS data from purvalanol A, which inhibits CDK.

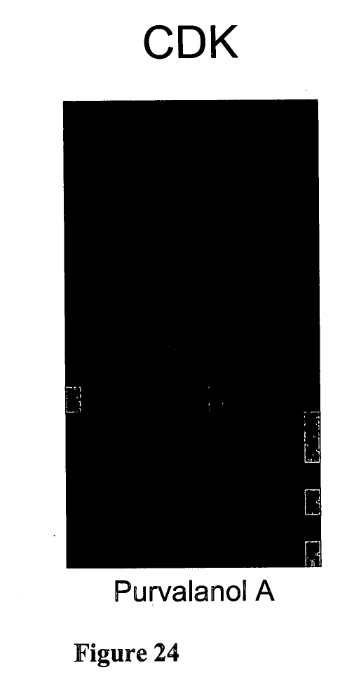

FIG. 25 shows the simple clustering of compounds listed on the right. Clustering provides a baseline for metric comparisons, is useful for evaluating reproducibility, replicates cluster reasonably well, and shows similar mechanism of action (e.g., tubulin).

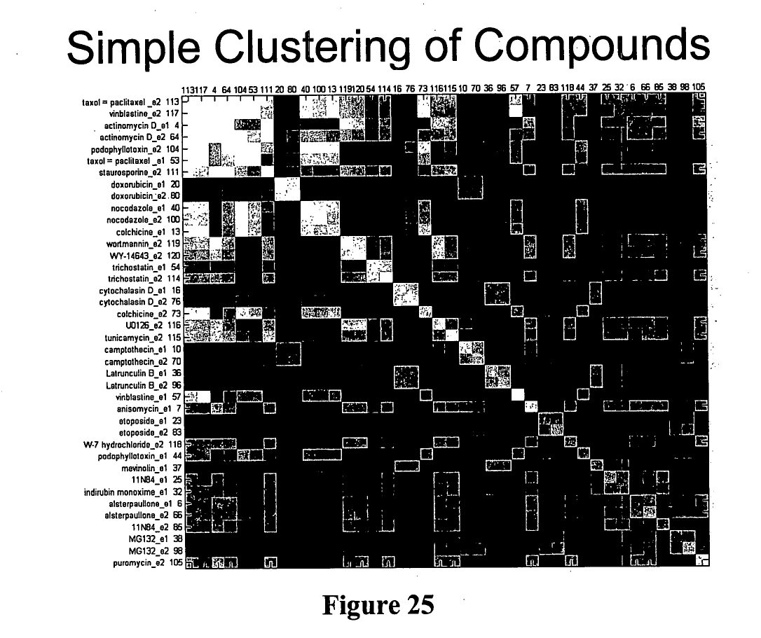

FIG. 26 shows the clustering of descriptors listed on the right-spliceosome average pixel area, spliceosome average grey, anillin average grey, spliceosome speckle count, DAPI average grey, DAPI pixel area, DAPI perimeter, DAPI perimeter, DAPI shape factor, and DAPI elliptic form factor. Clustering of descriptors is useful for evaluating descriptors, is useful for evaluating reproducibility, and replicates cluster reasonably well.

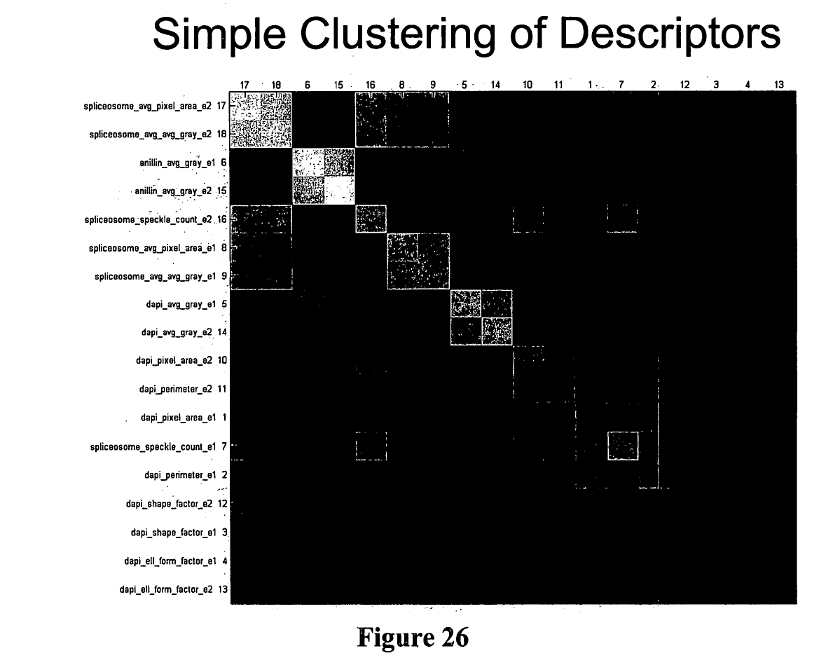

FIG. 27 shows more sophisticated clustering allowing for combing descriptors that are noise-tolerant, are dependent on relative concentration, and ignore absolute concentration. One way is by rank ordering the descriptors by concentration at which they undergo an inflection, noting if deflection is up or down.

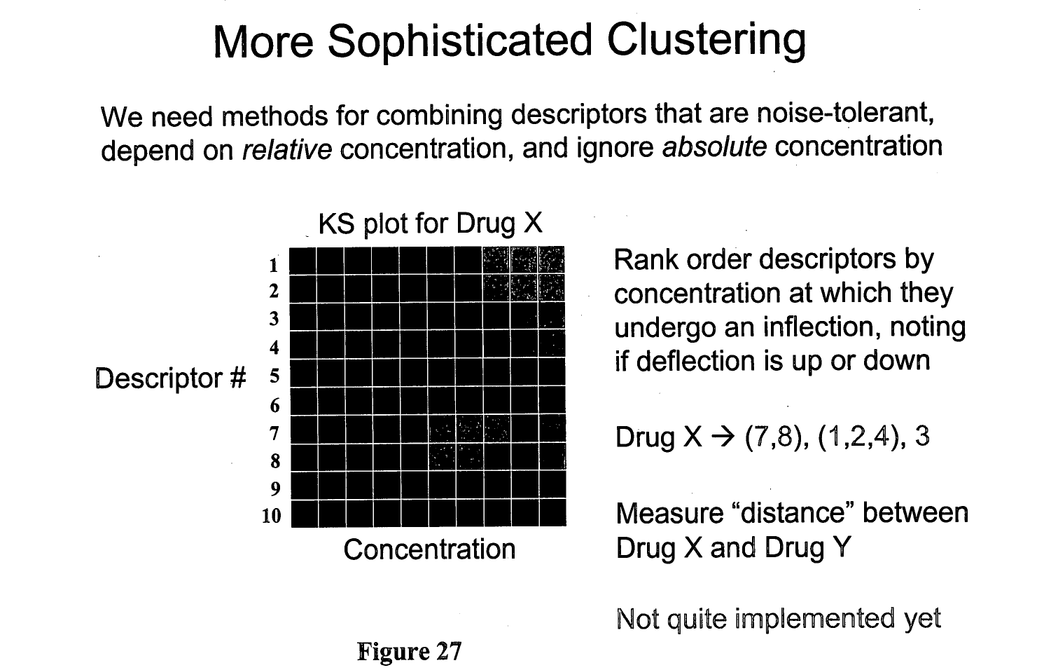

FIG. 28 shows clustering based on similar mechanisms of action (e.g., actin, tubulin, ribosome, and cyclooxygenase).

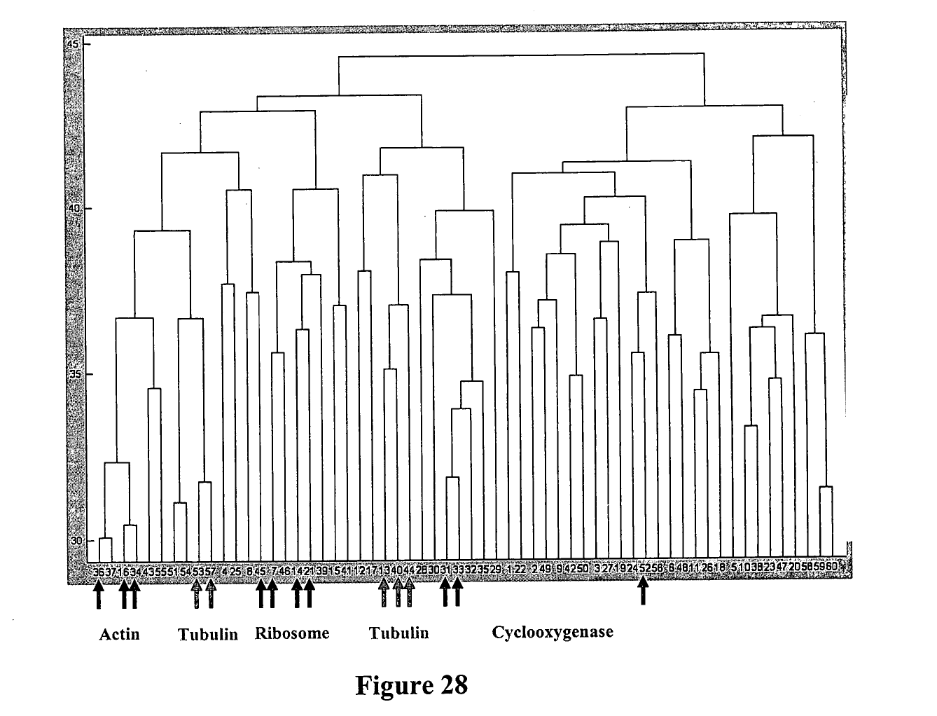

FIG. 29 shows analysis of clustering metrics by plotting percent true by total positives.

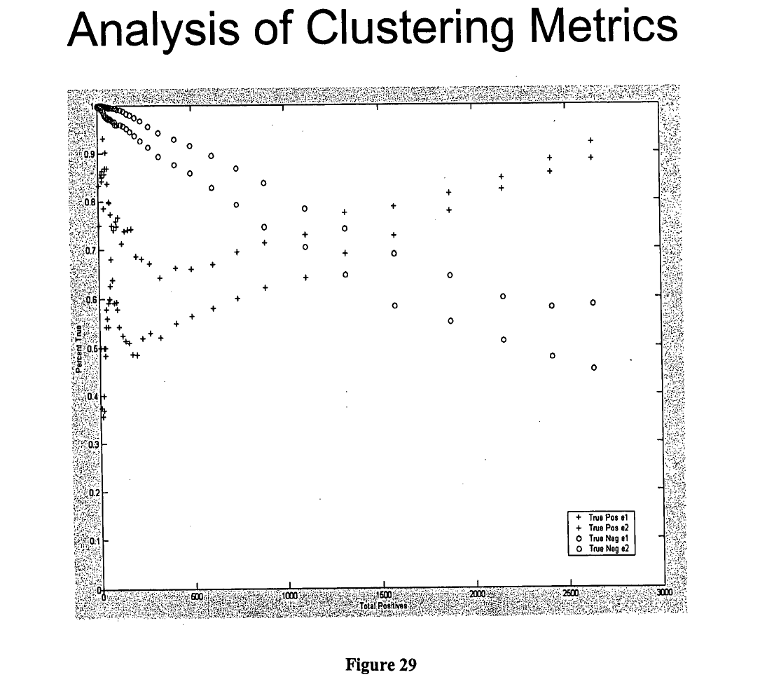

FIG. 30 shows analysis of clustering metrics by plotting percent true negatives by percent true positives.

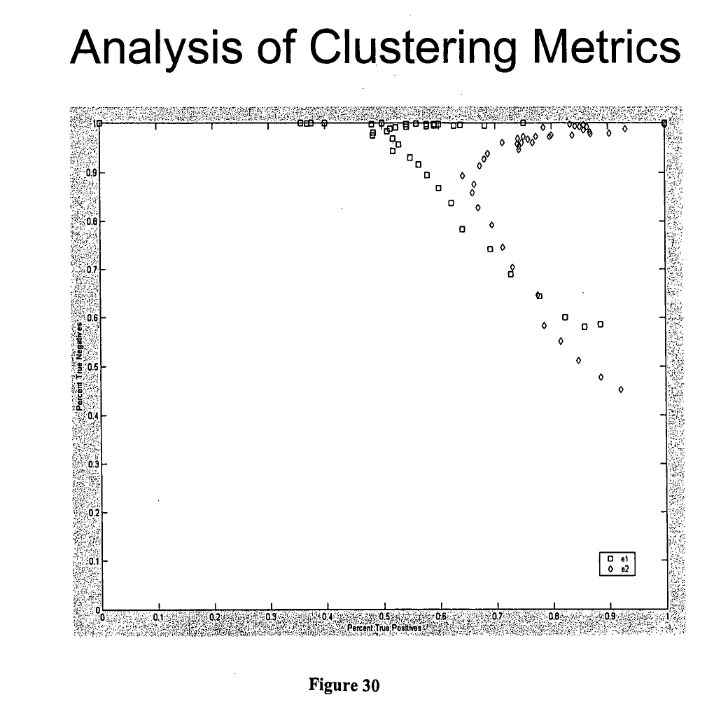

FIG. 31 shows the key steps in the algorithm for reducing image data to compound profile. A. Image segmentation. For each image (examples show DNA (blue), SC35 (red), and anillin (green)), we generate a nuclear region (blue) and a set of associated regions (shown here are cytoplasmic annulus (yellow) and SC35 speckles (green)). For each defined nuclear region, we measure multiple descriptors. B. Quantification of population response. For a given compound, titration, and descriptor, we generate a population histogram and related cumulative distribution function (cdf; black) to be compared against the control population (blue). Shown is a 3-fold dilution series ranging from 590 pM to 35 μM camptothecin. We reduce each experimental cdf, to a single dependent variable through comparison with a control population using non-parametric Kolmogorov-Smirnov (KS) statistic against a control population. Each vertical red or green line indicates the position and sign of the maximal height difference between the curves; this height is the KS statistic. C. Heat map of compound profile. A z-score is calculated for each KS statistic, and the vector of z-scores for all descriptors and all titrations is displayed for rapid visual assessment. Increased scores are represented in red, and decreased in green, with intensity encoding magnitude. Triangles to the right indicate descriptors shown in FIG. 31B and the triangle at bottom indicates the dose shown in FIG. 31A.

FIG. 32 is a comparison of compound profiles. As in FIG. 31C, the x axis shows increasing dose and the y axis encodes descriptors. Dose ranges are shown from 65 pM to 35 μM for all drugs except epothilone B, which is shown from 0.65 pM to 0.35 μM. The color scale is as in FIG. 31C. For ease of visualization, descriptors in all profiles are sorted in decreasing order of camptothecin response. (A) Compounds of similar mechanism show similar profiles. Shown are representative compound profiles. HDAC, histone deacetylase; ALLN, N-acetyl-Leu-Leu-norleucinal. (B) Compound profiles can distinguish differences between drugs with similar mechanisms.

FIG. 33 shows the hierarchical clustering of the 61 most responsive compound profiles by TISS values. Compound stock concentrations are in parentheses (FIG. 37). The left panel shows mechanism of compound as described in the literature. In blue are compounds that were blinded or are of unknown mechanism. The middle panel shows the matrix of P values derived from pairwise TISS values. The dendrogram at right shows the degree of association.

FIG. 34 is a single-cell analysis showing differing patterns of dose-dependent p53 and cFos responses to different drugs. A. Scatter plot of average nuclear p53 intensity vs. average cFos intensity in a typical control well and representative image. The bright cells at the top of the image are in mitosis. B. Dose-dependent increases in response to MG132 shown in heat maps are correlated in scatter plots and images (orange nuclei). C. Dose-dependent increases in response to camptothecin shown in heat maps are anti-correlated in scatter plots and images. The black (cFos) and green (p53) heat map values for the highest dose reflect the contribution of apoptotic cells with negligible p53 and cFos nuclear staining.

FIG. 35 shows a compound vector. A. The Kolmogorov-Smirnov statistic. Descriptors are measured on populations of treated (black curves on graphs) and untreated cells (blue curves on graphs). The Kolmogorov-Smirnov (KS) statistic, a non-parametric comparison of response, is defined as the difference of the two cumulative distributions computed at the position where the absolute difference between the two curves reaches its maximum (red and green lines respectively indicate positive and negative shifts of the descriptor measurements). The KS values are normalized by a measurement of the descriptor's variability and converted to z-scores (represented as red and green blocks respectively, indicating high and low z-scores; Supplemental text, section C). Compound vectors are made up of descriptor measurements taken over multiple titrations. B. Schema for compound vectors. We show an example of a compound vector X_{c} for a compound determined by three descriptors (descriptors 1-3 indicated by purple, black, and blue arrows) over four titrations.

FIG. 36 shows a determination of compound similarity. A. Shift-correlations of compound vectors. Shown are two compound vectors X₁ and X₂. It can be seen that X₂ is similar to X₁ except that its effect starts at a later titration value and it has some “noise” in the value of the first descriptor at the first titration (leftmost red square). Below are shown the titration sub-series for X₁(s) and X₂(s) obtained by sequentially truncating descriptor values at different titrations. Correlations are computed for each pair X₁(s) and X₂(−s) and the values are shown schematically at the right (yellow for negative correlation, blue for positive correlation). B. Similarity scores for comparing compound vectors. The rightmost column shows a matrix representation of the pairwise correlations of all compound vectors over a range of shift parameters s. The column to the left shows the histograms of these matrices. For each shift s, a non-parametric similarity score φ_{if} is assigned to the correlation determined in A. by computing the fraction of the histogram that lies to the right of the correlation value. As an overall similarity measurement between compound vectors X₁ and X₂, we take the minimum similarity score over all shifts: φᵢⱼ=min{φᵢⱼ(s)} (indicated by the dashed box around the value 0.04).

FIG. 37 shows the full set of replicate averaged compound profiles and titrations. Each compound is shown with its first and second replicate, followed by its averaged response profile. Descriptors (y-axis) and titrations x-axis) are ordered as in FIG. 31C. Compound labels are given with stock solution concentrations in parenthesis as in Table 2. Thus, concentrations ranges are: (0.67) 4.4 pM to 0.23 uM; (1) 6.5 pM to 0.35 uM; (4.5) 29.3 pM to 1.6 uM; (10) 65 pM to 3.5 uM; (25) 162.5 pM to 8.8 uM; (33) 214.5 pM to 11.6 uM; (50) 325 pM to 17.5 uM; (197) 1.3 nM to 69 uM.

FIG. 38 is a determination of range for titration shifts. For S ranging from 1 to 10, we calculated the average reproducibility of the full set of compounds; y-axis is measurement of reproducibility, x-axis is titration sub-series index. Top panel shows S between 1 and 5; middle panel shows S between 5 and 10; bottom panel compares S=4, 5, and 6. Thus, the graph for S=0 measures how reproducibly truncated compounds (FIG. 36) are matched to their replicate experiment allowing no shifts. As expected, a sharp peak around x=0 is seen. For larger values of S, broader regions of reproducibility are seen as shifting will bring truncated (identical) compounds back into alignment.

FIG. 39 shows the clustering of descriptors. Top panel shows (symmetric) hierarchical clustering of the averaged descriptor vectors using the TISS. TISS scores are generated on the basis of similarity of descriptors over the 61 compounds chosen in FIG. 33. Grey scale shows p-value as indicated by color bar to the right of the panel. Middle and bottom panels indicate marker and feature of each descriptor. DNA descriptors are only selected from the SC35 and anillin plate.

### DEFINITIONS

An agent is any chemical compound being contacted with the cells being analyzed by cytological profiling. These chemical compounds may include biological molecules such as proteins, peptides, polynucleotides (DNA, RNA, RNAi), lipid, sugars, etc.), natural products, small molecules, polymers, organometallic complexes, metals, etc. In certain embodiments, the agent is a small molecule. In other embodiments, the agent is a nucleic acid or polynucleotide. In yet other embodiments, the agent is a peptide or protein. In other embodiments, the agent is a non-polymeric, non-oligomeric chemical compound.

The Kolmogorov-Smirnov statistic (Chakravarti, Laha, and Roy, (1967) Handbook of Methods of Applied Statistics, Volume I, John Wiley and Sons, pp. 392-394) is used to decide if a sample comes from a population with a specific distribution. The Kolmogorov-Smirnov (K-S) test is based on the empirical distribution function (ECDF). Given N ordered data points Y1, Y2, . . . , YN, the ECDF is defined as where n(i) is the number of points less than Yi and the Yi are ordered from smallest to largest value. This is a step function that increases by 1/N at the value of each ordered data point. An attractive feature of this test is that the distribution of the K-S test statistic itself does not depend on the underlying cumulative distribution function being tested. Another advantage is that it is an exact test (the chi-square goodness-of-fit test depends on an adequate sample size for the approximations to be valid). Despite these advantages, the K-S test has several important limitations: (1) it only applies to continuous distributions; (2) it tends to be more sensitive near the center of the distribution than at the tails; (3) perhaps the most serious limitation is that the distribution must be fully specified. That is, if location, scale, and shape parameters are estimated from the data, the critical region of the K-S test is no longer valid. It typically must be determined by simulation. Due to limitations 2 and 3 above, many analysts prefer to use the Anderson-Darling goodness-of-fit test. However, the Anderson-Darling test is only available for a few specific distributions. The Kolmogorov-Smirnov test is defined by: H0: the data follow a specified distribution; Ha: the data do not follow the specified distribution; Test Statistic: the Kolmogorov-Smirnov test statistic is defined as where F is the theoretical cumulative distribution of the distribution being tested which must be a continuous distribution (i.e., no discrete distributions such as the binomial or Poisson), and it must be fully specified (i.e., the location, scale, and shape parameters cannot be estimated from the data).

A peptide or protein comprises a string of at least three amino acids linked together by peptide bonds. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc.

Polynucleotide or oligonucleotide refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g. 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

Small molecule refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Examples of small molecules that occur in nature include, but are not limited to, taxol, dynemicin, and rapamycin. In certain other preferred embodiments, natural-product-like small molecules are utilized.

Titration refers to the concentration of an agent. In certain embodiments, titration refers to the final concentration of an agent added to a cell or a population of cells. In certain embodiments, a range of titrations for a particular agent is used in the inventive system. A titration may range, for example, from 1 pM to 100 mM; 10 pM to 1 mM; 100 pM to 100 μM; or 10 pM to 10 μM.

Titration-invariant similarity score (TISS) refers to any statistic used to compare the dose-response profiles of any two agents independent of the staring dose. In certain embodiments, the TISS between two agents is calculated by defining a titration sub-series for each agent to account for different possible starting concentrations, a correlation is then calculated for pairs of these sub-series, and a similarity measure derived from the strongest correlation over a determined range of sub-series is defined. In certain embodiments, descriptors are compared using TISSs.

### DETAILED DESCRIPTION OF PREFERRED EMBODIMENTS

The present invention provides for a system for analyzing various aspects of a cell or population of cells which can be visualized using microscopy. These phenotypic aspects of the cell may be quantified in certain embodiments. This data can then be analyzed later to derive various categories, correlations, or trends among different populations of cells which may have been treated in different ways (e.g., different drugs, different agents, different concentrations, different RNAi's, different time points). The inventive system comprises imaging the cells, and analyzing the acquired images for various phenotypic aspects of the cells. The phenotypic aspects of the cells in a population may be quantitated and statistically analyzed, and this data may be compared to data from a control set of cells or cells subjected to different conditions. The data can then be clustered to find cells of similar phenotypes in order to find compounds of a known activity or mechanism of action.

Cell samples. Any test sample containing cells may be evaluated using the inventive system. The cells may be specially prepared for light microscopy, or they may be imaged and analyzed with no special preparations. In certain embodiments, the cells are imaged while they are still alive and immersed in media or other suitable solutions. The media or solution may contain staining or dyeing agents to enhance the visualization of certain feature of the sample such as certain cell types, cellular organelles, connective tissue, nucleic acids, proteins, etc. The cell samples may be in individual culture dishes coated with a suitable substrate such as poly-lysine, or they may be in multiple well plates such as 8, 16, 32, 64, 96, or 384-well plates. In experiments in which arrays of cells are being analyzed, a multi-well plate is preferable as would be appreciated by one of skill in the art.

In other embodiments, the cell samples are prepared for light microscopy by fixing the cells to a slide and staining the samples using stains known in the art. In certain embodiments, chemical compounds known to stain a particular types of cells or cellular organelle are used in the preparation of the cells. These stains may be fluorescent under specific conditions (e.g., a specific wavelength). In certain embodiments, the stains are small molecule dyes such as DAPI (4′,6-diamidino-2-phenylindole), acridine orange, hydroethidine, etc. Other stains may include Acid Fuchsin, Acridine Orange, Alcian Blue 8GX, Alizarin, Alizarin Red S, Alizarin Yellow R, Amaranth, Amido Black 10B, Aniline Blue Water Soluble, Auramine O, Azure A, Azure B, Basic Fuchsin Reagent A.C.S., Basic Fuchsin Hydrochloride, Benzo Fast Pink 2BL, Benzopurpurin 4B, Biebrich Scarlet Water Soluble, Bismarck Brown Y, Brilliant Green, Brilliant Yellow, Carmine, Lacmoid, Light Green SF Yellowish, Malachite Green Oxalate, Metanil Yellow, Methylene Blue, Methylene Blue Chloride, Methylene Green, Methyl Green, Methyl Green Zinc Chloride Salt, Methyl Orange Reagent A.C.S., Methyl Violet 2B, Morin, Naphthol Green B, Neutral Red, New Fuchsin, New Methylene Blue N, Nigrosin Water Soluble, Nigrosin B Alcohol Soluble, Nile Blue A, Nuclear Fast Red, Oil Red O, Orange II, Orange IV, Orange G, Patent Blue, 4-(Phenylazo)-1-naphthalenamine Hydrochloride, Phloxine B, Ponceau G R 2R, Ponceau 3R, Ponceau S, Procion Blue HB, Prussian Blue, Pyronin B, Pyronin Y, Quinoline Yellow SS, Rhodamine 6G, Rhodamine B Base Alcohol Soluble, Rhodamine B O, p-Rosaniline Acetate Powder, Rose Bengal, Rosolic Acid, Saffron, Safranine O, Stilbene Yellow, Sudan I, Sudan II, Sudan III, Sudan IV, Sudan Black B, Sudan Orange G, Tartrazine, Thioflavine T TG, Thionin, Toluidine Blue O, Tropaeolin O, Trypan Blue, Ultramarine Blue, Victoria Blue B, Victoria Blue R, Xylene Cyanol FF, Xylene Cyanol FF, Alizarin, Alizarin carmine (for staining bone), Alizarin red S (sodium monosulfonate) monohydrate, Alum carmine, Amaranth, Arsenazo III, Basic red 2 (Cotton red; Gossypimine; Safranin A or O or Y), Bismark brown, Bromocresol green, Bromocresol purple, Bromophenol blue, Bromophenol red, Bromothymol blue, Calcein, Calcon (Eriochrome black B), Clayton yellow (Thiazole yellow), Coomassie blue (Brilliant blue), Cotton Red (Basic red 2; Gossypimine; Safranin A or O or Y), Cresol red sodium salt, Cupferron, 2′,7′-Dichloro fluorescein, Dicyanobis (1,10-phenanthroline)Iron, Diethyldithiocarbamic acid silver salt, 4,7-Diphenyl-1,10-phenanthroline-x.x-disulfonic acid diNa salt, Diphenylthiocarbazone, Dithizone, Eosin bluish, Eosin Y, Eriochrome black B (Calcon), Eriochrome black T, Eriochrome blue, Eriochrome blue black R, Eriochrome blue SE, Eriochrome gray SGL, Eriochrome red B, Erionglaucine (A), Erythrosin B, Fast Green FCF, Fuchsin acid, Fuchsin basic (Pararosaniline HCI), Gentian Violet, Gossypimine (Basic red 2; Cotton red; Safranin A or O or Y), Hematoxylin, Hydroxy Naphthol blue, Indigo blue pigment, Janus green B, Methyl orange, Methyl orange, Methyl red, Methyl thymol blue, Methyl violet B (Aniline violet; Dahlia violet B), Methyl violet base (Solvent violet 8), Methylene blue, Murexide indicator, Neutral red, Orange G, Orange IV, Owen's blue, Patent blue (Acid blue 1), Pararosaniline HCI (Basic fuchsin), Phenolphthalein, Phenol red, Phlorglucinol dihydrate, Pyronine Y (or G), Safranin, Safranin A or O or Y (Basic red 2; Cotton red; Gossypimine), Solvent violet 8 (Methyl violet base), Sudan III, Sudan IV, Thiazole yellow (Clayton yellow), Thymol blue, Thymolphthalein pH indicator 9.4-10.6, Wright's stain, Xylene cyanole FF, Chromotrope 2B, Chromotrop 2R, Clayton Yellow; Cochineal Red A, Congo Red, Coomassie® Brilliant Blue G-250, Coomassie® Brilliant Blue R-250, Cotton Blue, Crocein Scarlet 3B, Curcumin, Diazo Blue B, Eosin B, Eosin B Water Soluble, Eosin Y, Eriochrome Black A, Eriochrome Black T Reagent A.C.S., Eriochrome Blue Black R, Eriochrome Cyanine R, Erioglaucine, Erythrosin B, Ethyl Eosin, Ethyl Violet, Evans Blue, Fast Garnet GBC Base, Fast Garnet GBC Salt, Fast Green FCF, Fluorescein Alcohol Soluble U.S.P., Fluorescein Alcohol Soluble, Fluorescein Water Soluble, Hematoxylin, 8-Hydroxy-136-pyrenetrisulfonic Acid Trisodium Salt; Indigo Synthetic, Indigo Carmine, Indophenol Blue, Indulin Water Soluble, and Janus Green B. In other embodiments, the stains may include labeled or unlabeled antibodies specific for a particular protein or antigen such p53, p38, p43, fos, c-fos, jun, NF-κB, anillin, SC35, CREB, STET3, SAMD, FKHD, D4G, calmodulin, calcineurin, actin, microtubulin, ribosomal proteins, receptors, cell surface antigens such as CD4, etc. In other embodiments, stains for Golgi markers, endosomal markers (e.g., EA1), lysosomal markers (e.g., LAMP-1, LAMP-2), and mitochondrial markers are used.

The cell samples which can be analyzed using the inventive method can be derived from any source. The cells may be derived from any species of animal, plant, bacteria, fungus, microorganism, or single-celled organism. Examples of sources include E. coli, Saccharomyces cerevisiae, S. pombe, Candida albicans, C. elegans, Arabidopsis thaliana, rats, mice, pigs, dogs, and humans. In certain embodiments in which chemical compounds are being screened for biological activity in humans, the cells are of mammalian origin, preferably of primate origin and even more preferably of human origin. In certain embodiments, the cells are well-known experimental cell lines which have been characterized extensively and have been found to perform reproducibly under various experimental conditions. Examples of such cells lines include various bacterial and yeast cells lines, HeLa cells, COS cells, NCI 60 cells, and CHO cells. In certain embodiments, the cell line used for cytological profiling is the HeLa cell line. In other embodiments, the cell lines used is the NCI 60 cell line. In certain embodiments, the cells may be derived from known cell lines, cultures, or tissue/cell samples from surgical, pathological, or biopsy specimens. If the cells being analyzed are part of a specimen, the cells may be an integral part of an organ or tissue and therefore be surrounded by connective tissue, extracellular matrix, support cells such as fibroblasts, blood cells, etc., blood vessels, lymphatics, etc.

The cell used in the sample may be wild type cells or may have been altered. The genome of the cells may have been altered using techniques known in the art to enhance the expression of a gene, decrease the expression of a gene, delete a gene, modify a gene, etc. The cells may also be treated with various chemical agents (e.g., small molecules, pharmaceutical agents, chemical compounds, biological molecules, proteins, polynucleotides, anti-sense agents such as RNAi, etc.) known to have a specific biological effect such as, for example, cytochalasin D, jasplakinoldie, latrunculin B, 105D, colchicine, griseofulvin, podophyllotoxin, taxol, vinblastine, actinomycin D, staurosporine, camptothecin, doxorubicin, etoposide, anisomycin, emetine, puromycin, tunicamycin, anisomycin, mevinolin, wortmannin, trichostatin, ibuprofen, indomethacin, sulindac sulfate, alsterpaullone, indirubin monoxime, olomucine, purvalanol A, cycloheximide, or nocodazole. Any combination of genetic and/or chemical alterations may also be used. For example, the cells may be genetically engineered to stop the cells in the cell cycle, and then chemical compounds from a library of compounds may be added to the genetically altered cells to identify compounds which patch the genetic defect.

As discussed supra, the cell samples may be provided as arrays of cells—each element of the array representing a separate experiment in which the cells have been subjected to different conditions. For example, each well of a multi-well plate may be treated with a different test agent, different concentration, different temperature, or different time point to determine its effect on the cells. The cells may be treated with an agent in concentrations ranging from 0.1 pM up to 100 mM; preferably, 1 pM to 0.1 mM; more preferably 10 pM to 0.01 mM. The cells may be treated using 100-fold, 50-fold, 20-fold, 10-fold, 9-fold, 8-fold, 7-fold, 6-fold, 5-fold, 4-fold, 3-fold, or 2-fold dilution series. In certain embodiments, cells are treated with a titrations series ranging from 10 pM to 100 μM, 1 pm to 10 μM, 100 pm to 100 μM, 10 pm to 1 mM, 1 nM to 100 μM, or 10 pm to 100 nM. In certain embodiments, the titration series ranges over 1 order of magnitude, 2 orders of magnitude, 3 orders of magnitude, 4 orders of magnitude, 5 orders of magnitude, 6 orders of magnitude, 7 orders of magnitude, 8 orders of magnitude, 9 order of magnitude, or 12 orders of magnitude. In certain embodiments, the array of cells has at least one element containing cells which are untreated and therefore serve as a control. In certain embodiments, several elements of the array may serve as a control to enhance reliability and reproducibility. The cells may optionally be fixed and stained before images of the cells are acquired. In other embodiments, images of the cells may be obtained while the cells are alive. This allows the cells to be analyzed at later time points, or the cells may be further treated with agents.

Image acquisition. The cells to be analyzed using the inventive method are first imaged to obtain the raw data that will be analyzed to determine the phenotypic characteristics of the cells. The number of cells to be imaged may range from a single cell to less than 100 cells to less than 500 cells to over a thousand cells. In certain embodiments, the number of cells in a field to be imaged range from 100-200 cells, preferably approximately 200 cells. In certain embodiments, images with less than 10 cells are discarded. In other embodiments, images with less than 50 cells are discarded. Multiple images of the cells may be taken at different wavelengths to assess staining with different fluorescent dyes. Multiple images may also be taken in each well in order to reduce noise and increase reproducibility in the experiments. For example, five to ten images may be acquired in each well at different non-overlapping regions. The cells can be imaged using any method known in the art of light or fluorescence microscopy.

Images may be obtained digitally using a digital image capture device such as a CCD camera or the equivalent, or they may be obtained conventionally using standard film technology and then digitized from the film (e.g., using a scanner). In either case, the camera may be connected to a microscope. In a preferred embodiment, the images are acquired digitally by a CCD camera directly mounted to a microscope, thereby eliminating the additional step of digitizing an analog image.

The magnification chosen to image the cells may range from very low magnification 5× to very high magnification 5000×. In certain embodiments, the magnification ranges is 10×, 20×, 50×, 100×, 200×, 500×, or 1000 ×. As would be appreciated by one of skill in this art, the magnification would depend on various factors including the number of samples to be imaged, the number of cells per samples, and the aspects of the cells to be analyzed. For example, analysis for cell shape and morphology would typically require less magnification than imaging subcellular organelles such as the nucleus and centrosomes. In certain embodiments, the cells may be imaged at multiple magnifications in order to better assess several different aspects of the cells. In other embodiments, a magnification is chosen as a compromise between various competing factors so that the cells are only imaged once.

An appropriate resolution (pixels per image) of the digitized image must be selected, whether the images are originally acquired by digital means or are scanned from conventional micrographs. As will be understood by those of ordinary skill in the art, resolution is typically selected so that features of interest (e.g., whole cells, nuclei, or centromeres) comprise a sufficient number of pixels that their morphological characteristics (e.g., average diameter, area, perimeter, shape factor) may be determined with a sufficient accuracy at the selected magnification, while not exceeding available computing power and/or data storage. If a camera with very fine resolution (i.e., a large number of pixels per imaged frame) is not available, a higher magnification may be used. In such cases, more image frames may be acquired for each specimen in order to image a statistically significant number of cells.

In certain embodiments, the images are acquired using a digital camera mounted on a standard laboratory microscope. The images may then be stored and analyzed later by a computer, or they can be analyzed as they are acquired. Images may be stored in any appropriate file format, including lossy formats such as .jpg and .gif or lossless formats such as .tiff and .bmp. Alternatively, only analysis results may be stored.

Cell features may be identified using standard thresholding and edge detection techniques. Such techniques are described, for example, in U.S. Pat. No. 5,428,690 to Bacus et al., U.S. Pat. No. 5,548,661 to Price et al., and U.S. Pat. No. 5,848,177 to Bauer et al., all of which are incorporated by reference herein. Once the cell features have been identified by one of these methods, quantitative morphological data about each feature may be collected, such as area, perimeter, shape factor (commonly defined as the ratio of 4π(Area)/(Perimeter)²), aspect ratio, and gray level statistics (such as the average gray level and the standard deviation in the gray level for a particular feature).

Data Analysis. Once the images have been analyzed for the specific cell characteristics and the characteristics have been quantified, any statistical methods known in the art can be used to determine the differences between two sets of data. In certain embodiments, a distribution of cells with a certain characteristic from a particular experiment may be used in statistically analyzing the characteristic. In certain embodiments, a set of experimental data involving a specific drug, at a particular concentration, and at a certain time point will be compared to a set of control data where no drug has been added. In other embodiments, experimental data with a first agent may be compared to experimental data with a second agent; or one concentration versus another concentration; or one time point versus another. In certain embodiments, a titration series using one agent is compared to a titration series using no agent (control) or a second agent. In other embodiment, statistical analysis may be performed on more than two sets of data resulting in a 3-way, 4-way, 5-way, or multi-way analysis.

In certain embodiments, distributions are obtained for each set of data collected. In certain embodiments, it is convenient to represent with a single number each population of descriptor values in a given experimental well. Some of the characteristics desired in such a reduced measure include: (1) it must cope with non-normal distributions of descriptor values (e.g., bimodal distributions); (2) it must account for the fact that different descriptors have different levels of biological variability and experimental noise; (3) it must convert different types of measurement into a common unit for comparison; (4) it must be insensitive to descriptor parameterization; and (5) it must be insensitive to the precise quantitative relationship between antibody staining intensity and total amount of target per cell. Preferably, the reduced measure will have at least one of the desired characteristics.

In certain embodiments, two distributions may be compared by comparing the heights of the two distributions, the widths of the two distributions (e.g., the width at the base, the width at half-height), continuous distribution functions of the two distributions, etc. In comparing the continuous distribution functions, one can determine the maximum distance or displacement between the two curves (i.e., the Kolmogorov-Smirnov statistic), the integration or area between the two curves, the maximum height difference between the two curves, the intersection of the two curves, etc.

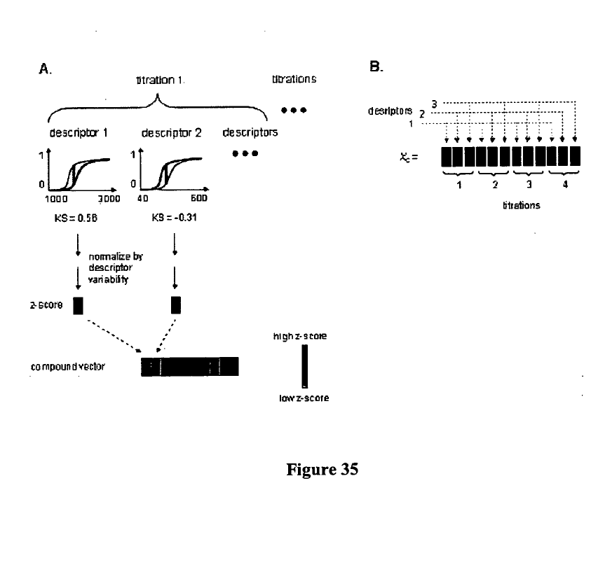

In certain embodiments, two sets of distribution data are compared using Kolmogorov-Smirnov statistics. Distributions of each data set are determined, and empirical cumulative distribution functions are calculated. The continuous distribution functions from each of the sets of data being compared are analyzed to determine the maximum displacement between the two cumulative distribution functions. That is, the function KS(f,g,) computes f−g at the point where |f−g| reaches its maximum. Note that KS(f,g,)=−KS(f,g,). The maximum displacement is a signed statistic known as the Kolmogorov-Smirnov statistic (KS statistics) (see FIG. 35). In certain preferred embodiments, one set of data is experimental (e.g., cells treated with a particular compound) and the other is a control (e.g., cells left untreated). The resulting KS statistics from multiple experiments can then be assigned a color and plotted in an array so that the KS statistics from many different experiments can be visually assessed.

As an example of computing a KS statistic, let f and g be the continuous distribution functions for nuclear area for cell in two wells—f represents cells from an untreated well and g represents cells from a treated well. If the average nuclear area were to increase in the treated well, then g would shift to the right (FIG. 35). This would result in KS(f,g,) being positive. If the nuclear size instead decreased in the treated well, then KS(f,g,) would become negative.

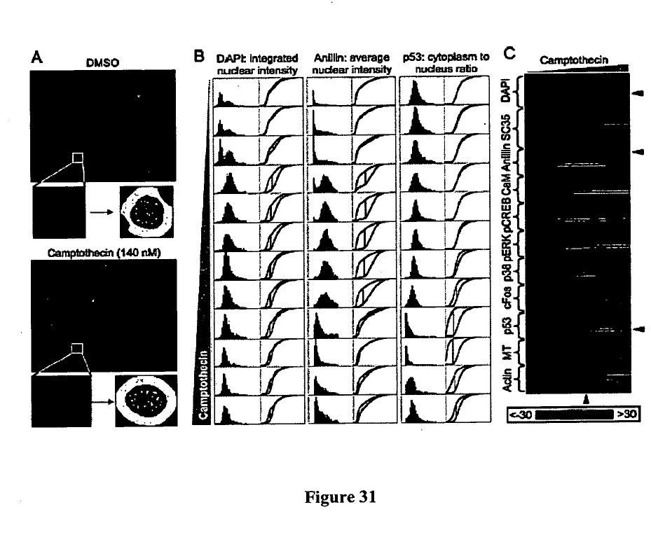

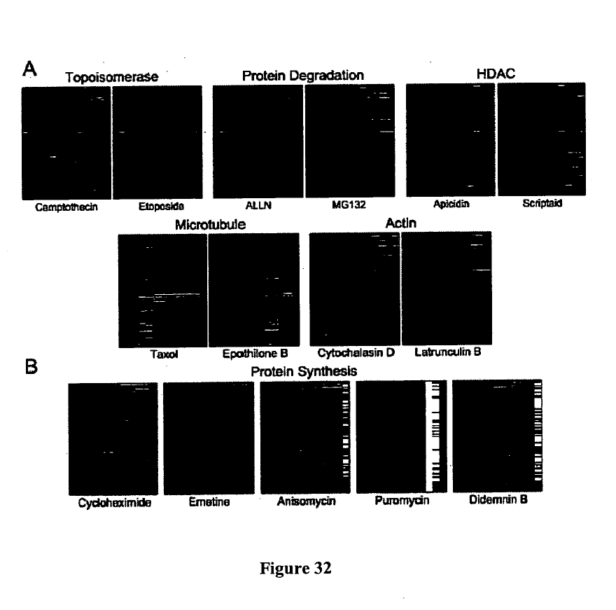

In a certain embodiment, in order to asses the effect of a test compound at a given titration, a KS statistic is computed for each descriptor. In certain embodiments the KS statistic is normalized to account for descriptor variability. The KS value may be normalized by any measurement of the descriptor's variability. For example a z-score may be calculated by dividing the KS statistics for a particular compound, titration, and descriptor (KS_{c,d,t}) by the standard deviation for the descriptor and population size in a control population (std(q_{d}(n)). The z-scores may then be assigned a color to generate a heat plot for easy visualization (see, e.g., FIGS. 31 and 32).

In other embodiments, the effect of an agent on a cell is complex. For example, the effect of the agent on a cell may be due to differential sensitivity of downstream pathways to degree of perturbation of a primary target. Or, the effect may be due to the binding of drug to multiple targets with different affinities. The similarity of test agents independent of the starting point of their titration series is assessed using a “titration-invariant” similarity score. The TISS between two test compounds is calculated as follows: (a) first a titration sub-series for each compound to account for different possible starting concentrations is defined; (b) a correlation for certain pairs of these sub-series within a range is defined; and (c) a similarity measure derived from the strongest correlation over a determined range of these sub-series is defined. In certain embodiments, cells are treated with a titrations series ranging from 10 pM to 100 μM, 1 pm to 10 μM, 100 pm to 100 μM, 10 pm to 1 mM, 1 nM to 100 μM, or 10 pm to 100 nM. The first step of calculating the TISS involves defining sub-series of Z-scores (as discussed above) by truncating starting or ending titrations thereby allowing one to “shift” the starting point for the titration series. In certain embodiments, the number of shifts scanned over is less than all possible shifts to reduce computational costs and reduce the changes of false identifications. In certain embodiments, the number of shifts scanned is 13, 12, 11, or 10. In other embodiments, the number of shifts scanned is less than 10, preferably 9, 8, 7, 6, 5, 4, or 3, most preferably 5. In certain embodiments, a greater than 500-fold range in titrations is scanned in each direction. In other embodiments, the range of titrations is approximately 100,000, 10,000, 1,000, 500, 450, 400, 350, 300, 350, 200, 150, 100, 50, or 10-fold. In the second step, for all pairs of compound vectors created in step one an s-correlation is determined. Last, one looks for the value of s in the correlation matrix created in step two that gives the highest correlation between the two vectors. The s-correlations may be normalized to provide for direct comparison of the s-correlations. Normalizing the s-correlations using a Gaussian distribution, a s-similarity score of 0 corresponds to the most correlated pair of compound vectors, and a s-similarity score of 1 corresponds to the least correlated pair of compound vectors. In certain other embodiments, the descriptor vectors are compared instead of compound vectors.

As would be appreciated by one of skill in this art, the reproducibility of these statistical calculations may be improved by analyzing a greater number of cells, for example, using replicates. In other embodiments, high and low values of a vector component may be dropped in calculating a replicate average to increase reproducibility.

Clustering algorithms can then be used to cluster data sets (e.g., compounds, descriptors) which are similar. In certain embodiments, standard hierarchical clustering algorithms are used. For example, clustering can be used to identify replicates of a compound within a set of data. Also, clustering can be used to cluster data from a compound with a known activity to data from a compound with a similar mechanism of action. In this way, the inventive system may be used to identify the mechanism of action of a new compound.

Clustering can also be used to better refine the cellular characteristics (descriptors) being evaluated. For example, clustering can be used to determine which descriptors can provide information that is independent or non-overlapping, or new correlations between descriptors.

Applications. Morphological analysis or cytological profiling of cells can be used in a wide variety of applications, for example, histology, pathology, drug screening, drug development, drug susceptibility screens, etc. In certain embodiments, chemical compounds are contacted with the cells, and the cells are imaged after a certain time period. In certain embodiments, different concentrations of the chemical compound dissolved in a suitable solvent such as medium, water, DMF, or DMSO are used. The cells are then imaged, and the data gathered from the images is analyzed to determine trends among different compounds or different descriptors.

In one embodiment, cytological profiling is used in drug discovery. First, a set of chemical compounds or drugs with known biological activity or mechanism of action, known as the training set, are contacted with cells at various concentrations and statistical data on various descriptors is gathered and analyzed. Trends are then established for certain compounds with known modes of action. For example, compounds that affect protein synthesis may affect certain descriptors while compounds that affect tubulin polymerization may affect other descriptors. After these trends have been established, a set of chemical compounds of unknown activities (e.g., a newly synthesized combinatorial library) may be contacted with the same cells to look for the affect of each of the compounds on the cytological profile of the cells. Clustering analysis comparing the training set of compounds to the new set of experimental compounds is then used to determine which compounds of unknown mechanisms of actions may have activities similar to compounds in the training set. Therefore, compounds more likely to have a desired activity can be quickly selected using cytological profiling.

System. The invention also provides a system for carrying out the inventive methods. The system may include some or all of the hardware and software necessary to practice the inventive technology. The system may include microscopes, microprocessors, data storage devices, robots, fluid handling devices, plate reader, automatic pipetters, software, printers, plotters, displays, etc. In certain embodiments, the system may include a microscope able to acquire images at various magnifications and/or resolutions, a microprocessor, and software for carrying out the image analysis and the statistical analysis of the raw data derived from the images. In certain embodiments, the system includes the hardware and/or software necessary to calculate Kolnogorov-Smirnov statistics. In certain embodiments, the system includes the hardware and/or software necessary to calculate titration-invariant similarity scores (TISSs). In other embodiments, the system includes the hardware and/or software necessary to perform clustering analysis. In certain embodiments, a low magnification is useful where many cells are to be analyzed. In other embodiments, a high magnification is useful when analyzing for a characteristic only visible at high power. In addition to magnification, the resolution of the image may be varied depending on the analysis to be performed. In certain embodiments, a low resolution image is preferred for carrying out the automated analysis. In certain embodiments, the system does not include the microscopy equipment needed to acquire the images. Instead, the raw data is analyzed by a system with a microprocessor running the necessary software for performing the desired analysis. For example, the system may run the necessary software for calculating K-S statistics, TISSs, or other statistics. The system may also include the necessary software for performing the clustering of compounds or descriptors. The system may also include a storage device for storing the images and/or data for future recall if need be.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Phenotypic Screening

To determine the reproducibility of cytological profiling, a set of 60 chemical compounds of known activity or mechanism of action were contacted with NCI 60 cells grown in 384-well plates. Each of the compound was administered to the cells at 16 different concentrations. After 20 hours, the cells were imaged by taking 4 images per well with a 20× objective (approximately 400 cells). Two imaging replicates and two full experimental replicates were obtained resulting in 8 images per well and 16 images for each compound/concentration combination. These images (approximately 120 GB of image date) were then used to extract approximately 6 GB of numerical data. These numerical data was then analyzed using statistical analysis such as K-S statistics and clustering to look for correlations and trends among the 60 compound tested. The data was also used to test the reproducibility and reliability of cytological profiling.

384-well plates were seeded with NCI 60 cells. One of 60 different compounds (the “training set”) at a varying concentrationc was added to each well of the plate. The compounds included cytochalasin D, jasplakinoldie, latrunculin B, 105D, colchicine, griseofulvin, podophyllotoxin, taxol, vinblastine, actinomycin D, staurosporine, camptothecin, doxorubicin, etoposide, anisomycin, emetine, puromycin, tunicamycin, anisomycin, mevinolin, wortmannin, trichostatin, ibuprofen, indomethacin, sulindac sulfate, alsterpaullone, indirubin monoxime, olomucine, purvalanol A, cycloheximide, or nocodazol. Each of the compound was dissolved in DMSO and administered to the cells at 16 different concentrations (serial 3× dilution). The cells were then incubated for 20 hours. An experimental replicate was performed for each well to improve reliability and test reproducibility.

After 20 hours, the cells were fixed and stained using DAPI (a fluorescent probe for DNA), a fluorescent probe for anillin, and a fluorescent probe for SC35. Eight images were obtained from each well. Each image contained approximately 200 cells, and images with less than 10 cells were discarded from the data set.

The images were then analyzed using MetaMorph imaging software (version 5.0) (Universal Imaging Corporation). Numerical values for nine descriptors were determined using MetaMorph. Nuclei as imaged by the DAPI stain were identified by thresholding. The morphological data collected for each identified nucleus were the area in pixels, the perimeter in pixel widths, the shape factor (4π(Area)/Perimeter²), the elliptic form factor (i.e., the aspect ratio, defined as the ratio of the maximum length to the breadth), and the average gray level of the pixels comprising the nucleus. For the stain for anillin, average gray was the descriptor. For the stain for SC35, speckle count, average speckle pixel area, and average speckle average gray were the descriptors. Distributions were determined for each descriptor with a particular compound at a particular concentration. Distributions were also calculated for the descriptors of the control images from the untreated wells. From the distributions, empirical cumulative distribution functions were calculated. The Kolmogorov-Smirnov statistic (the maximum displacement) was calculated for each experiment versus the control. The KS values were then assigned a color, and these colors for each descriptor was plotted against concentration in order to better visualize when changes were occurring for a particular compound. Clustering was then performed to identify replicates of a particular compound within a training set and to identify compound of a similar mechanism of action.

From the data obtained for the training set, one can predict the activity of compounds of unknown mechanism by comparing the K-S statistics of the training set with those of the new set of compounds. The experimental set of compounds is contacted with the cells, and the cells are imaged and analyzed as described above.

### Example 2

### Distinguishing Drug Mechanism using Automated Microscopy and Multi-Dimensional Dose-Response Profiling

In the context of drug discovery, profiling technologies are useful in measuring both drug action on a desired target in the cellular milieu and drug action on other targets. Ideally, such profiling should be performed as a function of drug concentration, since several factors make the effects of drugs highly dose-dependent. These include differential sensitivity of downstream pathways to degree of perturbation of a primary target, and binding of drugs to multiple targets with different affinities. In some cases, therapeutic mechanism may involve binding to more than one target with differing affinity (J. G. Hardman, L. E. Limbird, A. G. Gilman, Eds., The Pharmacological Basis of Therapeutics (McGraw-Hill, ed. 10, 2001); Marton et al., Nat. Med. 4:1293 (1998); each of which is incorporated herein by reference). To date, drug effects have been broadly profiled using transcript analysis, proteomics, and measurement of cell line-dependence of toxicity (Marton et al., Nat. Med. 4:1293 (1998); Weinstein et al., Science 275:343 (1997); Paull et al., Cancer Res. 52:3892 (Jul. 15, 1992); Scherf et al., Nat. Genet 24:236 (2000); Gunther et al., Proc. Natl. Acad. Sci. USA 100:9608 (2003); Leung et al., Nat. Biotechnol. 21:687 (2003); Lindsay, Nat Rev Drug Discov 2:831 (2003); Lum et al., Cell 116:121 (Jan. 9, 2004); Giaever et al., Proc. Natl. Acad. Sci. USA 101:793 (Jan. 20, 2004); Haggarty et al., J. Am. Chem. Soc. 125:10543 (Sep. 3, 2003); Root et al., Chem. Biol. 10:881 (September, 2003); each of which is incorporated herein by reference). In these studies, multi-dimensional profiling methods were only applied at a single drug concentration. The only studies in which drug dose were explicitly considered as a variable employed an essentially one-dimensional readout of phenotype, degree of cell proliferation (Weinstein et al., Science 275:343 (1997); Paull et al., Cancer Res. 52:3892 (Jul. 15, 1992); each of which is incorporated herein by reference). Two recent reviews have highlighted the possibility of using combinations of targeted phenotypic imaging screens to generate profiles of drug activity (Price et al., J. Cell Biochem. Suppl. 39:194 (2002); V. C. Abraham, D. L. Taylor, J. R. Haskins, Trends Biotechnol. 22:15 (January, 2004); each of which is incorporated herein by reference). Here, we suggest that large sets of unbiased measurements might serve as high-dimensional cytological profiles analogous to transcriptional profiles. We present a method based on hypothesis-free molecular cytology that provides multidimensional single-cell phenotypic information, yet is simple and inexpensive enough to allow extensive dose-response profiles for many drugs.

We assembled a test set of 100 compounds (Table 2): 90 were drugs of known mechanism of action, six were blinded alternate titrations from this set of known drugs, one (didemnin B) was a toxin reported to have multiple biological targets (M. D. Vera, M. M. Joullie, Med Res Rev 22:102 (March, 2002); incorporated herein by reference), and three were drugs of unknown mechanism. The known drug set was chosen to cover common mechanisms of toxicity or therapeutic action in cancer and other diseases, and to include several groups with a common target (macromolecule or pathway) but unrelated structures. We analyzed thirteen 3-fold dilutions of each drug, covering a final concentration range on cells from micromolar to picomolar. (Table 3 and Materials & Methods). HeLa (human cancer) cells were cultured in 384-well plates to near confluence, treated with drugs for 20 hrs, fixed, and stained with fluorescent probes for various cell components and processes. We chose 11 distinct probes that covered a range of cell biology, multiplexing a DNA stain and two antibodies per well (the probe sets are: (SC35, anillin), (α-tubulin, actin), (phospho-p38, phospho-ERK), (p53, cFos), (phospho-CREB, calmodulin)). Using automated fluorescence microscopy, we collected images of up to ˜8000 cells from each well. 26 wells on each plate were treated only with DMSO to generate a control population. The experiment was performed twice in parallel to provide a replicate dataset. Image segmentation procedures were used to automatically identify nuclei and nuclear organelles, and cytoplasmic regions were approximated as an annulus surrounding each identified nucleus (FIG. 31A). For each cell, region, and probe, a set of descriptors was measured. These included measures of size, shape, and intensity, as well as ratios of intensities between regions (93 descriptors total, Table S3). In all, ˜7×10⁷ individual cells were identified from >600,000 images, yielding ˜10⁹ data points.

We can examine the population response of each descriptor to increasing concentrations of a given drug, which we illustrate with the genotoxic compound camptothecin (C. J. Thomas, N. J. Rahier, S. M. Hecht, Bioorg Med Chem 12:1585 (Apr. 1, 2004); incorporated herein by reference) (FIG. 31B). At low concentrations, the histogram for the total DNA content has the characteristic bimodal shape reflecting a mixture of G1, S and G2/M cell populations. G2 and M populations may be distinguished by 2-dimensional display of total DNA signal against nuclear area (not shown). As drug concentration increases, the cells arrest with S/G2 DNA content (C. J. Thomas, N. J. Rahier, S. M. Hecht, Bioorg Med Chem 12:1585 (Apr. 1, 2004); incorporated herein by reference). The measured DNA content distribution shifts leftward as dose increases, and at the highest concentrations apoptosis is widely induced. Anillin, a cytokinesis protein whose levels reflect cell cycle progression (C. M. Field, B. M. Alberts, J. Cell Biol. 131:165 (October, 1995); incorporated herein by reference), shows marked nuclear accumulation in the G2 arrested state. p53, a transcription factor that is part of the genotoxic response pathway, is strongly induced at high camptothecin concentrations, but much less so at concentrations sufficient to promote G2 arrest.

For profiling studies, it is useful to reduce each population of descriptor values to a single number. Our study made several demands of this reduction: it must be able to compare distributions of arbitrary shape (FIG. 31B); it must be robust to variation in dynamic range and noise levels among different descriptors; it must convert different types of measurement into a common unit for comparison; it must be descriptor parameterization-independent (e.g., an intensity ratio should behave the same as its reciprocal); and it must be insensitive to the precise quantitative relationship between antibody staining intensity and antigen density. We devised a measure based on the Kolmogorov-Smirnov (KS) statistic, allowing nonparametric comparison of experimental and control distributions from the same plate (FIGS. 31B and 35). Dividing by a measure of the variability within the control population yielded a z-score, which can be displayed as a function of descriptor and drug concentration in a heat plot to allow rapid visual comparison of compound response profiles (FIG. 31C). These plots represent a family of dose response curves for a single drug, but differ from traditional curves reflecting changes in a biochemical measurement. In particular, the relationship between z-score and the original physical measure may be non-linear. For example, the statistically significant responses of p53 to low doses of camptothecin seen in FIG. 31C reflect subtle effects not easily discerned by eye in the source images.

The heat plots typically have a sharp transition, reflecting a concentration at which many descriptors become different from control values. We will refer to this as the primary effective concentration (PEC) for the drug. The isolated responses observed at some low concentrations represent noise that could be reduced by increasing replicates, improving experimental procedures, and normalizing for local variation in cell density. For 39 drugs, we saw no strong effect, leaving a heat plot dominated by noise. Those drugs either lack a target in HeLa cells, were used at inactive dosages, or effected changes not detectable with our antibody set. For nearly all of the 61 drugs that showed a strong response, some descriptors responded at concentrations other than the PEC (see examples in FIG. 32). This may reflect varying biological consequences of low and high saturation of a single target, or interactions with multiple targets with different affinities. For example, camptothecin binds primarily to DNA complexes with topoisomerase I, promoting DNA strand breaks and S-phase arrest at low concentrations, but also blocks transcription and a number of other cellular processes at higher concentrations (Thomas et al., Bioorg Med Chem 12:1585 (Apr. 1, 2004); incorporated herein by reference). Other drugs in our test set are known to have multiple targets, such as histone deacetylase inhibitors (Yoshida et al., Cancer Chemother. Pharmacol. 48(Suppl 1):S20 (August, 2001); incorporated herein by reference) and the general kinase inhibitor staurosporine (M. E. Noble, J. A. Endicott, L. N. Johnson, Science 303:1800 (Mar. 19, 2004); incorporated herein by reference), and were thus expected to show complex dose-response behavior. Such phenotypic complexity may help explain why toxicity at high doses is common even for therapeutic drugs that are apparently highly selective at the level of target binding.

Drugs with common targets reported in the literature but diverse chemical structures often showed similar profiles readily distinguished from those of drugs of different mechanism (FIG. 32A). In other cases, markedly different profiles were evident within a family, most notably the protein synthesis inhibitors (FIG. 32B). This may reflect different cell responses to alternative biochemical mechanisms of poisoning ribosomes (J. D. Laskin, D. E. Heck, D. L. Laskin, Toxicol Sci 69:289 (October, 2002); incorporated herein by reference) or perhaps the existence of significant alternate targets (M. D. Vera, M. M. Joullie, Med Res Rev 22:102 (March, 2002); incorporated herein by reference).

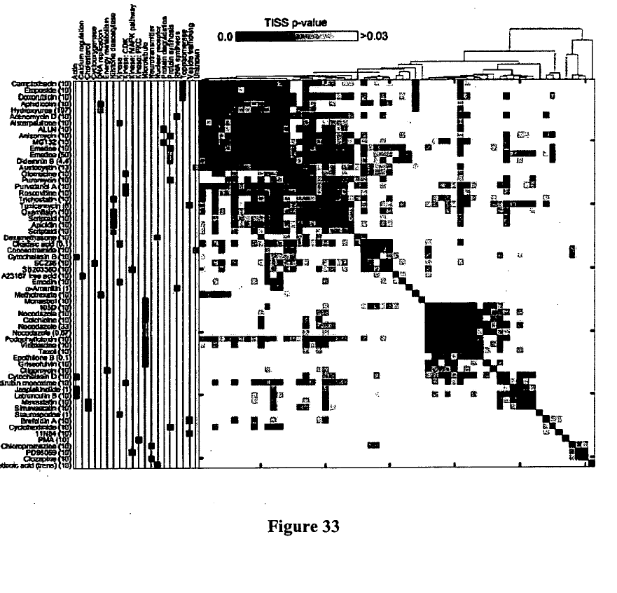

When comparing drug mechanism, changes in specificity, and thus phenotype, are relevant but changes in affinity, and thus PEC, are not. Two different dosage series of the same drug should result in similar heat plots shifted along the concentration axis. We developed a titration-invariant similarity score (TISS) to allow comparison between dose-response profiles independent of starting dose. TISS scores were generated for the 61 compounds that showed significant signal, and these were used for unsupervised clustering (FIG. 33). TISS was successful at grouping compounds with similar reported targets (Table 1).

TABLE 1
Assessment of TISS by literature categories.

| | | | Intensity, | | |
|---|---|---|---|---|---|
| | Full, KS | Intensity, | mean | #intra | #inter |
| Category | (pvalue) | KS (pvalue) | (pvalue) | pairs | pairs |
| Actin | 0.025 | 0.776 | 0.327 | 6 | 218 |
| DNA Replication | 0.011 | 0.057 | 0.007 | 3 | 168 |
| Histone Deacetylase | 0.001 | 0.024 | 0.489 | 10 | 265 |
| Kinase | 0.223 | 0.746 | 0.902 | 3 | 168 |
| Kinase CDK | 0.057 | 0.221 | 0.050 | 6 | 218 |
| Microtubule | 3.86E−20 | 9.81E−06 | 0.295 | 55 | 484 |
| Protein Synthesis | 6.02E−05 | 0.004 | 0.180 | 15 | 309 |
| Topoisomerase | 0.005 | 0.011 | 0.693 | 3 | 168 |
| Vesicle Trafficking | 0.206 | 0.314 | 0.514 | 3 | 168 |
| For each category having more than 2 compounds, we computed two sets of TISS scores: pair-wise TISS comparisons between members of the category and comparisons where only one element of the pair is in the category (columns 5 and 6 give these set sizes). As a crude in silico comparison to | | | | | |
| # other cell-based assays such as FACS (single-cell based) and cytoblots (whole population based), we repeated this procedure with a descriptor set comprising only total intensity measures, comparing with either our KS-based TISS scores or a mean-based TISS. P-values (columns 2-4) describe the probability that the rank ordering of the two sets of TISS values would have been seen by random draws from the same distribution. | | | | | |

As expected, clustering reflected biological mechanism rather than chemical similarity. For example, kinase inhibitors, most of which are ATP-mimetic compounds, did not cluster as a group. Clustering was poor even within a set of kinase inhibitors with overlapping targets (CDK inhibitors), perhaps reflecting variable inhibition of other kinases. The CDK inhibitors related by structure and reported target, purvalanol, roscovitine and olomucine, did cluster.

Of the blinded alternate titrations of known drugs, scriptaid, hydroxyurea, emetine, and two alternate series of nocodazole showed significant responses. These clustered closely with their unblinded counterparts and compounds of similar reported mechanism. Didemnin B, for which the reported range of activities includes inhibition of protein synthesis (M. D. Vera, M. M. Joullie, Med Res Rev 22:102 (March, 2002); incorporated herein by reference), clustered with ribosome inhibitors (see also FIG. 32B). Two of the three poorly characterized compounds showed strong responses. One, concentramide, is difficult to interpret. The other, austocystin, clusters with transcription and translation inhibitors. Preliminary experiments suggest that this compound inhibits transcription in vitro. Thus, our methods can group compounds of like mechanism and thereby suggest mechanism for new drugs.

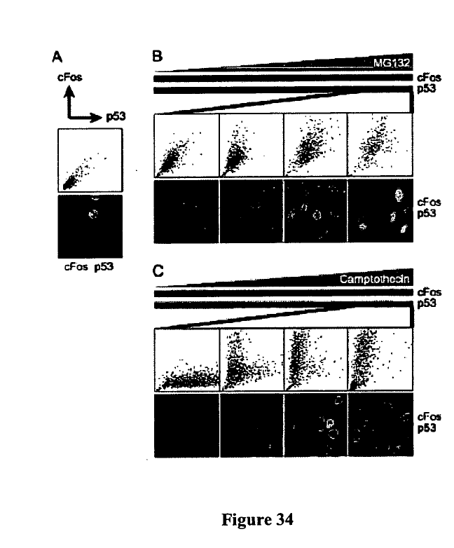

Extensions of cytological profiling to reflect dependencies among descriptors will allow more sophisticated analysis of drug responses at a systems level. For example, both p53 and cFos, a transcription factor involved in MAP-kinase signalling, are involved in cell stress responses, but the interrelationship of the p53 and MAP-kinase pathways is poorly understood (B. Kaina, Biochem Pharmacol 66:1547 (Oct. 15, 2003); incorporated herein by reference). Single-cell profiling reveals that different drug mechanisms induce different relative patterns of response by these two pathways (FIG. 34). The proteasome inhibitor MG132 causes increased correlated induction in these pathways, while responses to camptothecin are anti-correlated. Anti-correlated responses observed in fixed-time images may reflect switching of mutually exclusive cell states in response to different degrees of stress, or might reflect a dynamic temporal response, such as oscillation, that is not synchronized among cells (Lahav et al., Nat. Genet. 36:147 (February 2004); incorporated herein by reference). Using these data to establish a concentration/time window, live imaging will be required to distinguish between these hypotheses.

Cytometric dose-response profiling is a fast and cheap method for quantitatively surveying broad ranges of individual cell responses. We have used our methods to assign mechanism to blinded and uncharacterized drugs and to suggest systems-level relationships between signaling pathways. The complex dose-response curves and large cell-to-cell variability we frequently observed reinforce the utility of unbiased multidimensional characterization of drug effects over wide ranges of doses.

Many improvements and extensions of this work are possible. These include better lab automation, broader drug reference sets, different types of perturbation such as RNAi, improved strategies for cell segmentation, more sophisticated feature extraction (R. F. Murphy, M. Velliste, G. Porreca, Journal of Vlsi Signal Processing Systems for Signal Image and Video Technology 35:311 (November 2003); Conrad et al., Genome Res 14:1130 (June 2004); each of which is incorporated herein by reference), different sets of antibody probes and cells, the inclusion of more time points and live cell imaging, and the integration of complementary profiling strategies. Additionally, our methods may be extended to allow the characterization of responses by subpopulations defined by such variables as cell cycle state, cell density or neighboring environment. This analysis, extended to work in tissues or clinical samples, offers the potential to speed the identification of toxic compounds during therapeutic drug development and the targeting of drug effects to specific subtypes of cells.

Materials and Methods

A. Cell Culture and Immunofluorescence

Cell culture. 20 hours before compound addition, Hela cells grown in 150 mM dishes were trypsinized, resuspended in DMEM supplemented with 10% FCS and 10 ug/mL penicillin/streptomycin, and plated in 384-well plates at an initial density of 3,000 cells per well, 40 uL per well. Compounds were purchased from Sigma (St. Louis, MO), Calbiochem (EMD Biosciences, San Diego, Calif.) and Tocris (Elliksville, MO) and are listed in Table 2. Compound stocks were prepared in DMSO and then arrayed on 384-well plates in 16 consecutive 1:3 serial dilutions in DMSO as outlined in Table 3. The highest and two lowest dilutions (rows A, 0 and P) were not used for subsequent analysis as the wells due to persistent edge effects, leaving us with a series of 13 dilutions. Each stock was diluted 16-fold in warmed culture medium, and 8 μl of this solution was added to the plated cells, resulting in a 96-fold final dilution. All conditions were performed in duplicate in separate plates. Cells were incubated at 37° C. for 20 hours, then fixed in 3% formaldehyde in PBS. All liquid handling was performed using a programmed TekBench (TekCel, Hopkinton, Mass.).

TABLE 2
Compounds used for profiling. Compounds 91-100 were blinded during
method development.

| | | | | Used in |
|---|---|---|---|---|
| | | [ ]_{stock} | | clustering |
| Cpd# | Name | (mM) | Major activity | (FIG. 33) |
| 1 | 105D | 10 | Microtubule | Y |
| 2 | A23187 free acid | 10 | Calcium regulation | Y |
| 3 | Amanitin | 1 | RNA | Y |
| 4 | Actinomycin D | 10 | RNA | Y |
| 5 | ALLN | 10 | Protein degradation | Y |
| 6 | Alsterpaullone | 10 | Kinase | Y |
| 7 | Anisomycin | 10 | Protein synthesis | Y |
| 8 | Brefeldin A | 10 | Vesicle trafficking | Y |
| 9 | 8-bromo-cAMP | 10 | Kinase; PKA | |
| 10 | Camptothecin | 10 | Topoisomerase | Y |
| 11 | Chelerythrine | 10 | Kinase; PKC | |
| 12 | Ciglitazone | 10 | Nuclear receptor | |
| 13 | Colchicine | 10 | Microtubule | Y |
| 14 | Cycloheximide | 10 | Protein synthesis | Y |
| 15 | Cyclosporin A | 10 | Calcium regulation | |
| 16 | Cytochalasin D | 10 | Actin | Y |
| 17 | Deoxymannojirimycin | 10 | Vesicle trafficking | |
| 18 | Deoxynorjrimycin | 10 | Vesicle trafficking | |
| 19 | Dexamethasone | 10 | Nuclear receptor | Y |
| 20 | Doxorubicin | 10 | Topoisomerase | Y |
| 21 | Emetine | 10 | Protein synthesis | Y |
| 22 | Emodin | 10 | Kinase | Y |
| 23 | Etoposide | 10 | Topoisomerase | Y |
| 24 | Exol | 10 | Vesicle trafficking | |
| 25 | 11N84 | 10 | Vesicle trafficking/ | Y |
| | | | kinase | |
| 26 | Forskolin | 10 | Kinase; PKA | |
| 27 | Genistein | 10 | Kinase | |
| 28 | Griseofulvin | 10 | Microtubule | Y |
| 29 | H89 | 10 | Kinase | |
| 30 | Hydroxyurea | 30 | DNA Replication | |
| 31 | Ibuprofen | 10 | Cyclooxygenase | |
| 32 | Indirubin monoxime | 10 | Kinase; CDK | Y |
| 33 | Indomethacin | 10 | Cyclooxygenase | |
| 34 | Jasplakinolide | 1 | Actin | Y |
| 35 | Lactacystin | 1 | Protein degradation | |
| 36 | Latrunculin B | 10 | Actin | Y |
| 37 | Mevastatin | 10 | Cholesterol | Y |
| 38 | MG132 | 10 | Protein degradation | Y |
| 39 | Monastrol | 10 | Microtubule | Y |
| 40 | Nocodazole | 10 | Microtubule | Y |
| 41 | Okadaic acid | 0.1 | Kinase | Y |
| 42 | Olomucine | 10 | Kinase; CDK | Y |
| 43 | PMA | 10 | Kinase; PKC | Y |
| 44 | Podophyllotoxin | 10 | Microtubule | Y |
| 45 | Puromycin | 10 | Protein synthesis | Y |
| 46 | Purvalanol A | 10 | Kinase; CDK | Y |
| 47 | Rapamycin | 10 | Kinase; PI3K | |
| | | | pathway | |
| 48 | Retinoic acid (trans) | 10 | Nuclear receptor | Y |
| 49 | Roscovitine | 10 | Kinase; CDK | Y |
| 50 | ICRF193 | 10 | Topoisomerase | |
| 51 | Staurosporine | 1 | Kinase | Y |
| 52 | Sulindac sulfide | 10 | Cyclooxygenase | |
| 53 | Taxol | 10 | Microtubule | Y |
| 54 | Trichostatin | 10 | Histone deacetylase | Y |
| 55 | Tunicamycin | 6 | Vesicle trafficking | Y |
| 56 | U0126 | 10 | Kinase; MAPK | |
| | | | pathway | |
| 57 | Vinblastine | 10 | Microtubule | Y |
| 58 | W-7 hydrochloride | 10 | Calcium regulation | |
| 59 | Wortmannin | 10 | Kinase; PI3K | |
| | | | pathway | |
| 60 | WY-14643 | 10 | Nuclear receptor | |
| 1 | Cytochalasin B | 10 | Actin | Y |
| 62 | Chloropromazine | 10 | Neurotransmitter | Y |
| 63 | PD98059 | 10 | Kinase; MAPK | Y |
| | | | pathway | |
| 64 | Clozapine | 10 | Neurotransmitter | Y |
| 65 | Trifluoperazine | 10 | Neurotransmitter | |
| 66 | SB202190 | 10 | Kinase; MAPK | |
| | | | pathway | |
| 67 | LY294002 | 10 | Kinase; PI3K | |
| | | | pathway | |
| 68 | Sodium butyrate | 10 | Histone deacetylase | |
| 69 | Nitropropionate | 10 | Energy metabolism | |
| 70 | Simavastatin | 10 | Cholesterol | Y |
| 71 | Niflumic acid | 10 | Cyclooxygenase | |
| 72 | Fluobiprofen | 10 | Cyclooxygenase | |
| 73 | Fluoxetine | 10 | Neurotransmitter | |
| 74 | Scriptaid | 10 | Histone deacetylase | Y |
| 75 | SC560 | 10 | Cyclooxygenase | |
| 76 | Apicidin | 10 | Histone deacetylase | Y |
| 77 | Epothilone B | 0.1 | Microtubule | Y |
| 78 | Oxamflatin | 10 | Histone deacetylase | Y |
| 79 | SC236 | 10 | Cyclooxygenase | Y |
| 80 | SB203580 | 10 | Kinase; MAPK | Y |
| | | | pathway | |
| 81 | Aphidicolin | 10 | DNA Replication | Y |
| 82 | PD169316 | 10 | Kinase; MAPK | |
| | | | pathway | |
| 83 | Methotrexate | 10 | DNA Replication | Y |
| 84 | Ceramide | 10 | Kinase; PKC | |
| 85 | Leupeptine | 10 | Protein degradation | |
| 86 | Sodium azide | 10 | Energy metabolism | |
| 87 | Zvad | 1 | Protein degradation | |
| 88 | CKI7 | 10 | Kinase | |
| 89 | TPEN | 10 | Metal homeostasis | |
| 90 | Oligomycin | 10 | Energy metabolism | Y |
| 91 | Nocodazole | 33 | Microtubule | Y |
| 92 | Nocodazole | 0.67 | Microtubule | Y |
| 93 | Indomethacin | 25 | Cyclooxygenase | |
| 94 | Hydroxyurea | 197 | DNA Replication | Y |
| 95 | Filopodine | 36 | Unknown | |
| 96 | Emetine | 50 | Protein synthesis | Y |
| 97 | Scriptaid | 10 | Histone deacetylase | Y |
| 98 | Didemnin B | 4.5 | Protein synthesis/ | Y |
| | | | Unknown | |
| 99 | Austocystin | 13 | Unknown | Y |
| 100 | Concentramide | 10 | Unknown | Y |

TABLE 3
Plate design.
Concentration dependence:

| Row | |
|---|---|
| A | [ ]_{stock} |
| B | [ ]_{stock}/3 |
| C | [ ]_{stock}/9 |
| D | [ ]_{stock}/27 |
| E | [ ]_{stock}/81 |
| F | [ ]_{stock}/2.4E+2 |
| G | [ ]_{stock}/7.3E+2 |
| H | [ ]_{stock}/2.2E+3 |
| I | [ ]_{stock}/6.6E+3 |
| J | [ ]_{stock}/2.0E+4 |
| K | [ ]_{stock}/5.9E+4 |
| L | [ ]_{stock}/1.8E+5 |
| M | [ ]_{stock}/5.3E+5 |
| N | [ ]_{stock}/1.6E+6 |
| O | [ ]_{stock}/4.8E+6 |
| P | [ ]_{stock}/1.4E+7 |

Compound distribution:

| Column | Plate 1 | Plate 2 | Plate 3 | Plate 4 | Plate 5 | Plate 6 |
|---|---|---|---|---|---|---|
| 1 | DMSO | DMSO | DMSO | DMSO | DMSO | DMSO |
| 2 | 1 | 21 | 41 | 61 | 81 | DMSO |
| 3 | 2 | 22 | 42 | 62 | 82 | DMSO |
| 4 | 3 | 23 | 43 | 63 | 83 | DMSO |
| 5 | 4 | 24 | 44 | 64 | 84 | DMSO |
| 6 | 5 | 25 | 45 | 65 | 85 | DMSO |
| 7 | 6 | 26 | 46 | 66 | 86 | DMSO |
| 8 | 7 | 27 | 47 | 67 | 87 | DMSO |
| 9 | 8 | 28 | 48 | 68 | 88 | DMSO |
| 10 | 9 | 29 | 49 | 69 | 89 | DMSO |
| 11 | 10 | 30 | 50 | 70 | 90 | DMSO |
| 12 | DMSO | DMSO | DMSO | DMSO | DMSO | DMSO |
| 13 | DMSO | DMSO | DMSO | DMSO | DMSO | DMSO |
| 14 | 11 | 31 | 51 | 71 | 91 | DMSO |
| 15 | 12 | 32 | 52 | 72 | 92 | DMSO |
| 16 | 13 | 33 | 53 | 73 | 93 | DMSO |
| 17 | 14 | 34 | 54 | 74 | 94 | DMSO |
| 18 | 15 | 35 | 55 | 75 | 95 | DMSO |
| 19 | 16 | 36 | 56 | 76 | 96 | DMSO |
| 20 | 17 | 37 | 57 | 77 | 97 | DMSO |
| 21 | 18 | 38 | 58 | 78 | 98 | DMSO |
| 22 | 19 | 39 | 59 | 79 | 99 | DMSO |
| 23 | 20 | 40 | 60 | 80 | 100 | DMSO |
| 24 | DMSO | DMSO | DMSO | DMSO | DMSO | DMSO |

Markers. Five sets of markers were stained by standard immunofluorescence methods in this study. The marker sets are α-tubulin (DM1α, Sigma) and actin (TxRed phalloidin, Sigma); SC35 (Sigma) and anillin (Gift from Christine Field, Harvard Medical school); phospho-p38 (pThr180/pTyr182, Sigma) and phospho-ERK (PT115, Sigma); p53 (BP53-12, Sigma) and cFos (Sigma); phospho-CREB and calmodulin (Upstate Signaling, Lake Placid, N.Y.). Hoechst 33342 (Sigma) was included in all marker sets to label nuclei.

Automated fluorescence imaging. Images were acquired using a NikonTE300 inverted fluorescence microscope equipped with an automated filter wheel (Sutter), motorized x-y stage (Prior), piezoelectric-motorized objective holder (Physik Instrumente), cooled CCD camera (Hamamatsu), and robotic plate-transfer crane (Hudson), all controlled by Metamorph software (Universal Imaging) (J. C. Yarrow, Y. Feng, Z. E. Perlman, T. Kirchhausen, T. J. Mitchison, Comb Chem High Throughput Screen 6:279-86 (2003); each of which is incorporated herein by reference). The α-tubulin/actin and SC35/anillin marker sets were imaged with a Plan Fluor 20× objective and 1×1 camera binning, the p-p38/p-ERK and p53/cFos marker sets were imaged with a Plan Fluor 20× objective and 2×2 camera binning, and the p-CREB/CaM marker sets were imaged with a Plan Fluor 10× objective and 2×2 camera binning. Nine images were acquired for each well.

B. Image Processing and Descriptor Extraction

Image analysis was performed on a 50 node Linux cluster running Matlab 6.5, Image Processing Toolkit 3.2.

Background subtraction. We determine the background intensities for each image by using the Matlab imopen function to perform a grayscale opening with a disk of radius 40 pixels (1×1 binning) or 20 pixels (2×2 binning). The subtraction of this background image from the original is used in all further processing.

Region segmentation. Nuclear definition. To maximize robustness to variation in staining and illumination intensity, as well as to minimize the need for assumptions about cell size and shape, we use a rapid segmentation approach that relies solely on the sign of the second derivative of intensity. In contrast to the more conventional use of the second derivative as part of an edge-detection strategy, we take advantage of the convexity of nuclear intensity at low resolutions and directly identify discrete regions of negative valued second-derivative. DNA intensity images are convolved with a Laplacian-of-a-Gaussian of width 1.5 pixels (1×1 binning) or 0.75 pixels (2×2 binning). This filtered image is thresholded for values less than −1, and holes in the resulting regions are filled using the Matlab imfill command. Nucleolar definition. The holes filled during the generation of nuclear regions, which correspond to small Regions of positive curvature, are defined as nucleoli. Spliceosome definition. SC35 Images are convolved with a Laplacian-of-a-Gaussian of width 1 pixel and discrete Regions with values less than −60 are identified. The intersection of these regions with Each nuclear region is determined. Cytoplasm definition. Each nuclear region is dilated By a disc of radius 14 pixels (1×1 binning) or 7 (2×2 binning) and the difference of this region with the set of all nuclear regions is determined.

Descriptors. For each nuclear region and associated cytoplasm, nucleolar and spliceosome regions, a set of descriptors are measured as described in Table 4.

TABLE 4
Descriptors extracted from images.

| Marker sets | # | Descriptor | Comment |
|---|---|---|---|
| A. DNA | 1 | Area | Pixel area of nuclear region |
| | 2 | Eccentricity | Ratio of axes of the best ellipse fit to nuclear |
| | | | region |
| | 3 | Perimeter | Area in pixels of nuclear region |
| | | | boundary returned by Matlab primitive |
| | | | bwperim |
| | 4 | Shape | Factor 4π Area/(Perimeter)2 |
| | 5 | Total Intensity | Integrated intensity in nuclear region |
| | 6 | Average Intensity | Average intensity in nuclear region |
| | 7 | Intensity | Variance of intensity in nuclear region |
| | | Variance | |
| | 8 | Gray Scale | Distance in pixels between grayscale |
| | | Centroid Offset | and binary centers of mass for nuclear |
| | | | region |
| | 9 | Solidity | Ratio of area of the nuclear region to the |
| | | | area of its convex hull |
| B. actin, | 1 | Total Intensity | Integrated intensity in nuclear region |
| anillin, cFos, | | | |
| CaM, pCREB, | | | |
| pERK, p38, | | | |
| p53, α-tubulin | | | |
| | 2 | Average Intensity | Average intensity in nuclear region |
| | 3 | Variance in | Variance of intensity in nuclear region |
| | | Intensity | |
| | 4 | Cytoplasm Area | Pixel area of annular cytoplasm region |
| | 5 | Average | Average intensity in cytoplasm region |
| | | Cytoplasm | |
| | | Intensity | |
| | 6 | Average | Ratio of B.5/B.2 above |
| | | Cytoplasm | |
| | | Intensity/Average | |
| | | Nuclear Intensity | |
| | 7 | Nuclear | Ratio of B.2/A.6 above |
| | | Intensity/DNA | |
| | | intensity | |
| | 8 | Gray Scale | Offset Distance in pixels between |
| | | Centroid | grayscale and binary centers of mass for |
| | | | nuclear region |
| C. SC35 | 1-8 | Same as B.1-7 | Same as B.1-7 |
| | 9 | Speckle Area | Total area of speckle regions |
| | 10 | Average Speckle | Average intensity of speckle regions |
| | | Intensity | |
| | 11 | Variance in | Variance in intensity of speckle regions |
| | | Speckle Intensity | |
| | 12 | Speckle Count | Number of discrete speckle regions |
| | | | (using Matlab “4-neighborhoods”) |

C. Data Analysis

The image processing and descriptor extraction described above resulted in the identification of 7×10⁷ regions and ˜10⁹ parameters from >620,000 images, leading to a collection of 30,000 empirical cumulative distribution functions (cdf's). We will refer to these cdf's below as p_{c,d,t}, where c is a compound index (Table 2), d is a descriptor index (Table 3), and t is a titration index (1 through 13).

Kolmogorov-Smirnov non-parametric statistics. A single image might contain cells in many different states, so spatially resolved cell measurements can produce data distributions that are difficult to reduce to simple parametric models. For example, even an untreated population contains cells spread throughout the cell cycle, so measurements of nuclear area are not drawn from a normal distribution.

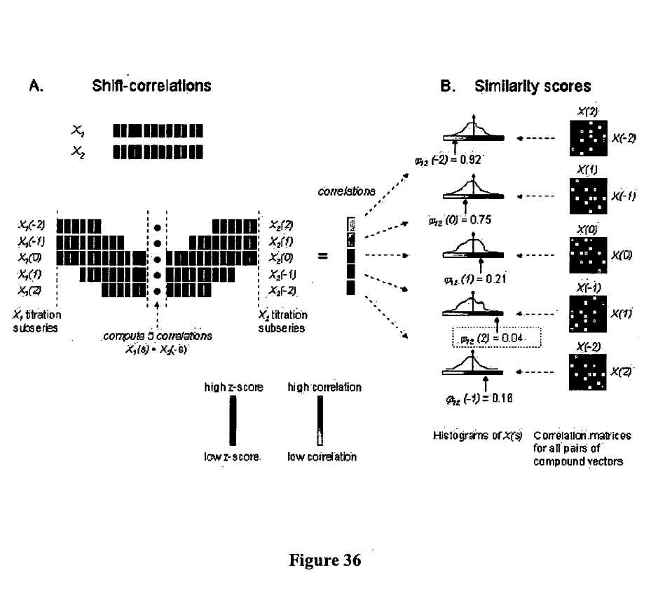

We make repeated use in our analysis of a standard non-parametric method for comparing cdf's, the Kolmogorov-Smirnov (KS) statistic (S2, 3) (FIG. 36). The function KS(f,g) computes f−g at the point where |f−g| reaches its maximum. Note that KS(f,g)=−KS(g,f).

As an example, let f and g be the cdf's of nuclear areas measured in two wells, f from an untreated well and g from a treated well. If the average nuclear area were to increase in the treated well, then the cdf of g would shift to the right (FIG. 35). This would result in KS(f,g) becoming positive. If the nuclear size were instead to decrease, then KS(f,g) would become negative.

Measurement of cytometric changes. As described in section A, each 384-well plate had 64 wells of control (DMSO-treated) cells; 26 DMSO wells, interior to the plate, were chosen to build a control population in subsequent analysis (rows B-O, columns 12 and 13). The total number of control cell nuclear regions varied per plate from 174,309 to 204,922 for the plates imaged at 10× and from 50,923 to 96,583 for the plates imaged at 20×. We wanted to obtain 1) an estimate of the plate variability of each descriptor d and 2) an estimate of the dependence of this variability on sample size. To do this, we drew (with replacement) 100 random subpopulations at each of 20 selected population sizes n between 100 and 20,000. We generated KS statistics for each subpopulation by comparing its cdf with the cdf of the remaining controls cells. For each descriptor and population size, we calculate the std_{d}(n), providing a measure of a descriptor's variability on untreated cells. We linearly interpolated std_{d}(n) between the 20 chosen values of n. Note that for every descriptor, we expect the mean of the KS stats to be ≈0.

In order to assess the effect of a compound c at a given titration t, we compute for each descriptor d the KS statistic KS_{c,d,t}=KS_{c,d,t}(p_{c,d,t}, q_{f}), providing a quantitative measurement of a population response p_{c,d,t} compared with the control population q_{d}. In order to assign a significance to the KS_{c,d,t} values and to normalize for descriptor variability, we compute z-scores by z_{c,d,t}=KS_{c,d,t}/std(q_{d}(n)), where n is the population size of the cells used to determine P_{c,d,t}. In the case of missing data (<100 cells per well) a z-score of zero is assigned.

Titration-invariant similarity score (TISS) for comparing descriptor and compound vectors. We developed a “titration-invariant” similarity score (TISS) to assess the similarity of compounds independent of the starting point of their titration series. The TISS between two compounds is calculated in three steps: (1) we define the notion of a titration sub-series for each compound to account for different possible starting concentrations (FIGS. 35 and 36B); (2) we define a correlation for pairs of these sub-series (FIG. 36B); (3) we define a similarity measure derived from the strongest correlation over a determined range of these sub-series (FIG. 36B).

(1) For each compound c, the complete set of z-scores across all descriptors and titrations defines a DxT-dimensional vector: X_{c}=(z_{c,l,l}, . . . , z_{c,D,l}, . . . , z_{c,l,T}, . . . , z_{c,D,T}), where D is the number of descriptors (=93) and T is the number of titrations (=13). In order to allow comparisons of compounds with different titration starting points, we define titration sub-series as follows: X_{c}(s)=(z_{c,l,l}, . . . , z_{c,D,l}, . . . , z_{c,l,T-s}, . . . , z_{c,D,T}−s) and X_{c}(−s)=(z_{c,l,s}, . . . , z_{c,D,s}, . . . , z_{c,l,T}, . . . , z_{c,D,T}). Intuitively, by truncating starting or ending titrations, these definitions allow us to “shift” the starting point for the titration series.

(2) For all compound vectors Xi and Xj, we define their s-correlation:
xᵢⱼ(s)=<Xᵢ, Xⱼ>(s)=<Xᵢ(s), Xⱼ(−s)>/(∥Xᵢ(s)∥ ∥Xⱼ(−s)∥)

(we use the standard notation <A, B>=ΣᵢAᵢBᵢ and ∥A∥²=<A,A>). Thus, <Xᵢ, Xⱼ>(0) measures the standard correlation of vectors Xᵢ and Xⱼ, while <Xᵢ, Xⱼ>(1) drops the first titration for compound Xⱼ and the last for Xᵢ before measuring their correlation. For each s, we built a 200×200 such correlation matrix X(s)=(xᵢⱼ(s)) using all of the compounds from each of the two replicates.

(3) Given a range −S≦s≦S, we wish to look for the value of s that gives the highest correlation between two vectors. Since the s-correlations of compound vectors are not directly comparable for different values of s, we used a non-parametric ranking to normalize these values. The 40,000 entries in each matrix followed an approximate Gaussian distribution (data not shown) and were used to define an s-similarity score: φᵢⱼ(s)=(# entries in X(s)≦(Xᵢⱼ(s)−1)/40,000. Thus, s-similarity scores of 0 and 1 correspond respectively to the most and least correlated pairs of compound vectors. The TISS between two compound vectors is then defined to be their highest correlation over all truncations φᵢⱼ=min{φᵢⱼ(s)}. Below we describe how we chose S, the range of allowable shifts s.

Note that the entire discussion above can be directly applied to descriptor vectors Y_{d}=(z_{l,d,l}, . . . , z_{ld,T}, . . . , z_{C,d,l}, . . . , z_{C,d,T}), where C is the total number of compounds. Hence, descriptor vectors may also be compared.

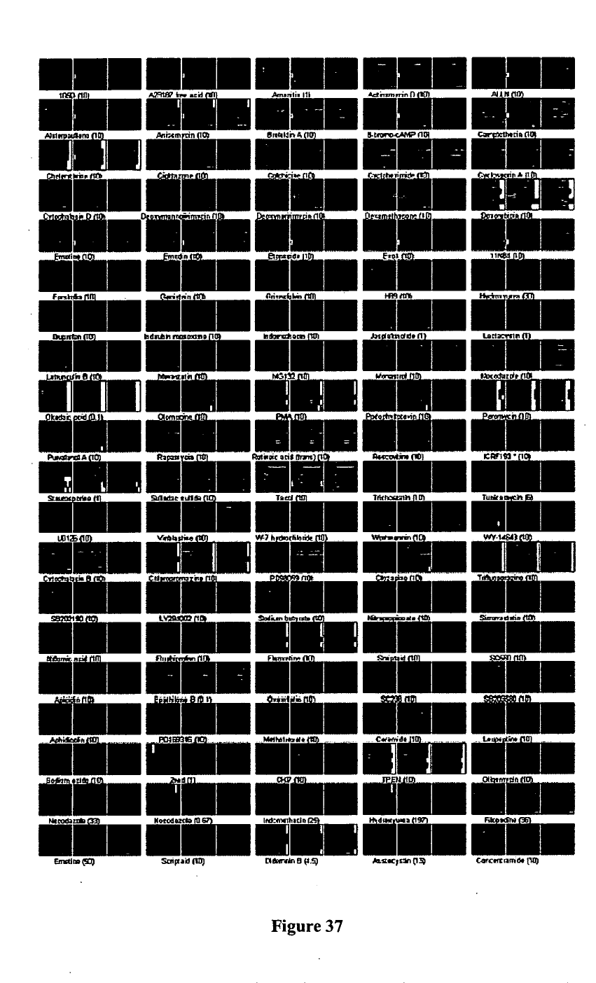

In subsequent discussions, when we refer to a “replicate averaged” (descriptor or compound) vector, we mean: take both experimental replicates of the vector and average their components (FIG. 37). In the case where data are missing from one component, we will take the other value. If both values are missing, we define the value to be zero (this case happened <1% of the time). 6 of our 50 compound plates showed pervasive imaging artefacts, and in these cases only one replicate was used (the plates dropped for the averaging process are: SC35/anillin plate 2, replicate 2; p-CREB/CaM plate 4, replicate 2; p-p38/p-ERK plate 1, replicate 2 and plate 2, replicate 1; α-tubulin/actin plate 1, replicate 2 and plate 4, replicate 2).

Measurement of reproducibility. We developed a scoring method to assess whether a compound vector carries reliable distinguishing information. For a given compound vector X_{c}, we calculate reproducibility by measuring its TISS with every other compound vector, including both experimental replicates. We define the measurement of reproducibility R(X_{c}) to be the percentage of compound vectors less similar to X_{c} than to its experimental replicate. A measurement of 1 indicates perfect reproducibility, i.e. X_{c} is more similar to its replicate than any other compound vector. A reproducibility score for a collection of compound vectors is taken to be the average of R evaluated on each member of the collection. This measurements may also be defined for a descriptor vector X_{d}, and is denoted R(X_{d}).

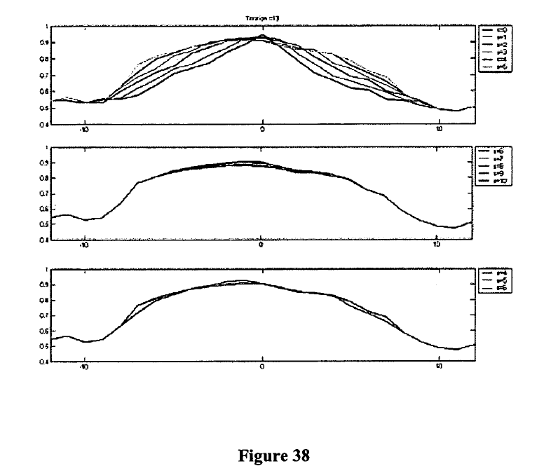

Choosing the range S of allowable shifts. In practice, we do not want to scan over all possible shifts (13 in either direction) when looking for titration invariant effects as it increases both computational cost and increases the chance of false identifications. For S ranging from 1 to 10, we calculated the average reproducibility of the full set of compounds. We determined that S=5 is a desirable range as 1) it provided an acceptable reproducibility score (>80%) over a 5-fold (=243-fold) range of titrations in each direction, 2) it did not significantly degrade the reproducibility compared with S <5, and 3) it gave similar results to S>5 (FIG. 38).

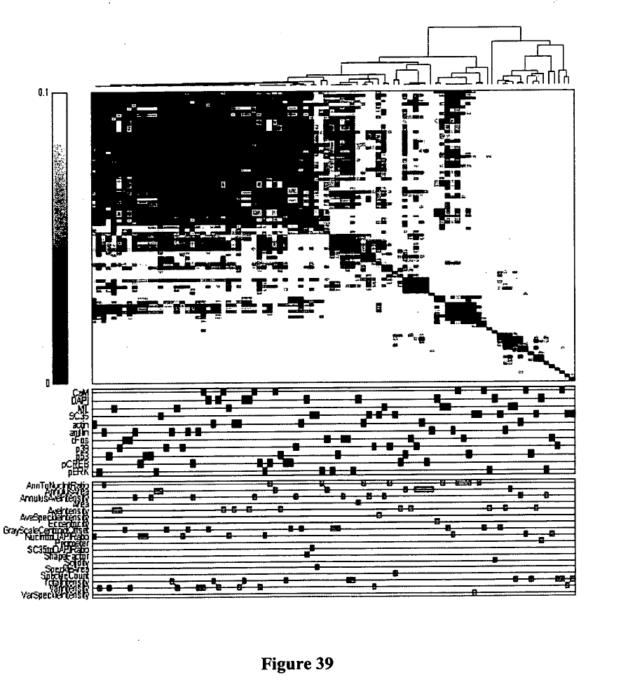

Clustering compound and descriptor vectors. We performed standard hierarchical clustering of replicate-averaged compound (FIG. 33) or descriptor (FIG. 39) vectors using the pdist and linkage functions in Matlab. pdist was defined by the TISS for each pair of vectors. Compound clustering was restricted to the 61 compounds that showed response above a signal threshold set to exclude >80% of control compound vectors generated from plate 6. Compound 50 was not present in all datasets and so was also excluded.

We note that other clustering approaches are possible. Significant progress has been made toward categorizing protein distributions in unperturbed cells (R. F. Murphy, M. Velliste, G. Porreca, Journal of Vlsi Signal Processing Systems for Signal Image and Video Technology 35:311 (November, 2003); Conrad et al., Genome Res. 14:1130-6 (2004); each of which is incorporated herein by reference), and this work may become applicable as larger reference sets are established and as we develop a better understanding of the range of categories of drug mechanisms and the characteristics of cell phenotypes that best represent these categories.

Assessment of TISS by literature categories. We tested the ability of TISS to discriminate between categories defined by literature-based mechanistic annotation (Table 1). For each category having more than 2 compounds, we computed two sets of TISS scores: pair-wise TISS comparisons between members of the category (intra-set, Table 1 column 5) and comparisons where only one element of the pair is in the category (inter-set, Table 1 column 6). To test the separation of these two distributions, we employed the nonparametric Wilcoxon rank sum test. The p-values shown in column 2 describe the probability that the rank ordering of the two sets of TISS values would have been seen by random draws from the same distribution.

As a crude in silico comparison of our ability to discriminate among these functional categories using data that would be available from such other cell-based assays as FACS (single-cell based) and cytoblots (B. R. Stockwell, S. J. Haggarty, S. L. Schreiber, Chem Biol 6:71-83 (1999); incorporated herein by reference) (whole population based), we reduced our descriptor set to only those based on total intensity measures. In our simulation of the FACS assay, we made full use of our statistical techniques (Table 1, column 3) whereas for the cytoblot simulation, we replaced our z-score based on the KS test with a z-score based on the difference of the means of the experimental and control population intensity values (Table 1, column 4). The resulting ability to discriminate among categories in both cases was significantly reduced.

TABLE 5
Descriptor sort order for FIG. 32

CaM_AnnToNucIntRatio
pERK_AnnToNucIntRatio
pCREB_AnnToNucIntRatio
anillin_AnnToNucIntRatio
p38_AnnToNucIntRatio
SC35_AnnToNucIntRatio
p53_AnnToNucIntRatio
p38_AnnulusAveIntensity
actin_AnnulusAveIntensity
SC35_AnnulusAveIntensity
cFos_AnnToNucIntRatio
actin_VarIntensity
actin_AveIntensity
p38_GrayScaleCentroidOffset
pERK_GrayScaleCentroidOffset
MT_VarIntensity
DNA_Eccentricity
DNA_VarIntensity
actin_AnnToNucIntRatio
SC35_GrayScaleCentroidOffset
DNA_AveIntensity
MT_AnnulusAveIntensity
anillin_GrayScaleCentroidOffset
MT_AveIntensity
p53_GrayScaleCentroidOffset
anillin_AnnulusAveIntensity
CaM_GrayScaleCentroidOffset
cFos_GrayScaleCentroidOffset
MT_AnnToNucIntRatio
cFos_AnnulusAveIntensity
DNA_GrayScaleCentroidOffset
actin_NucInttoDNARatio
p53_AnnulusAveIntensity
pERK_AnnulusAveIntensity
actin_GrayScaleCentroidOffset
p38_VarIntensity
DNA_ShapeFactor
pCREB_AnnulusAveIntensity
MT_NucInttoDNARatio
pCREB_GrayScaleCentroidOffset
cFos_AveIntensity
MT_GrayScaleCentroidOffset
CaM_AnnulusAveIntensity
cFos_NucInttoDNARatio
p38_NucInttoDNARatio
SC35_SC35toDNARatio
p38_AveIntensity
actin_TotalIntensity
SC35_AveIntensity
SC35_VarSpeckleIntensity
SC35_VarIntensity
SC35_SpeckleCount
p53_AveIntensity
SC35_SpeckleArea
DNA_Solidity
SC35_AveSpeckleIntensity
cFos_VarIntensity
CaM_NucInttoDNARatio
p53_NucInttoDNARatio
cFos_AnnulusArea
p53_AnnulusArea
cFos_TotalIntensity
DNA_TotalIntensity
MT_TotalIntensity
p53_VarIntensity
SC35_AnnulusArea
anillin_AnnulusArea
anillin_AveIntensity
anillin_VarIntensity
pERK_NucInttoDNARatio
anillin_NucInttoDNARatio
p53_TotalIntensity
DNA_Perimeter
p38_TotalIntensity
CaM_VarIntensity
DNA_Area
SC35_TotalIntensity
pERK_AnnulusArea
p38_AnnulusArea
anillin_TotalIntensity
CaM_AveIntensity
pERK_VarIntensity
MT_AnnulusArea
actin_AnnulusArea
pCREB_VarIntensity
pCREB_NucInttoDNARatio
pCREB_AveIntensity
CaM_AnnulusArea
pCREB_AnnulusArea
pERK_AveIntensity
pERK_TotalIntensity
pCREB_TotalIntensity
CaM_TotalIntensity

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A method of cell analysis, the method comprising steps of:
providing cells for analysis;
contacting the cells with at least two agents over a range of titrations;
imaging the cells;
analyzing images of the cells for various visual characteristics;
quantitating the visual characteristics of the cells;
calculating a Kolmogorov-Smirnov statistic for a particular agent, titration, and descriptor as compared to untreated control cells based on a continuous distribution function of the quantitated visual characteristic;
calculating z-scores by normalizing the Kolmogorov-Smirnov statistic for all descriptors and titrations based on the variability of the quantitated visual characteristic;
defining a titration sub-series by shifting the starting point of the titration series over a range of possible shifts;
calculating an s-correlation for each pair of titration sub-series for two agents; and
determining the value of s that yields the highest correlation between two titration subseries.

2. The method of claim 1, wherein the step of determining further comprises normalizing the s-correlations using a Gaussian distribution.

3. The method of claim 1 further comprising:
clustering of agents based on the s-correlation.

4. The method of claim 1, wherein the characteristic is selected from the group consisting of eccentricity of cells, average number of nuclei per cell, average area of cells, average volume of cells, average number of centromeres per cell, average size of nuclei, average area of nuclei, average size of cells, perimeter of cell, perimeter of nucleus, average gray value of staining, degree of staining, pattern of staining, ratio of staining between nucleus and cytoplasm, and morphology.

5. The method of claim 1, wherein the step of calculating z-scores comprises dividing the Kolmogorov-Smirnov statistic by the standard deviation calculated for each descriptor based on a control, untreated population.

6. The method of claim 1, wherein the titrations are within the range of 1 pM agent to 10 mM agent.

7. The method of claim 1, wherein the titrations are within the range of 10 pM agent to 100 μM.

8. The method of claim 1, wherein the number of titrations is at least 5.

9. The method of claim 1, wherein each titration represents a 2-fold dilution.

10. The method of claim 1, wherein each titration represents a 3-fold dilution.

11. The method of claim 1, wherein each titration represents a 5-fold dilution.

12. A method of screening, the method comprising steps of:
providing a plurality of cell samples;
providing a plurality of test agents;
contacting one of the cell samples with one of the test agents over a range of titrations;
imaging the plurality of cell samples after a time period;
analyzing the images of the cell samples for various visual characteristics (descriptors);
quantitating the data for each descriptor, agent, and titration;
calculating a Kolmogorov-Smirnov statistic for a particular descriptor, agent, and titration as compared to untreated, control cells based on a continuous distribution function;
calculating z-scores by normalizing the Kolmogorov-Smirnov statistic for all sets of descriptors, agents, and titrations based on the variability of the descriptor;
defining a titration sub-series by shifting the starting point of the titration series over a range of possible shifts;
calculating an s-correlation for each pair of titration sub-series for two agents; and
determining the value of s that yields the highest correlation between two titration subseries.

13. The method of claim 12, wherein the step of determining further comprises normalizing the s-correlations using a Gaussian distribution.

14. The method of claim 12 further comprising clustering of agents based on the s-correlation.

15. The method of claim 12, further comprising selecting those test agents that achieve a certain characteristic of the cells upon exposure of the cells to the test agent.

16. The method of claim 12, wherein the plurality of cell samples comprises greater than 100 cell samples.

17. A method of calculating a titration-invariant similarity score, the method comprising steps of:
providing numerical data quantitating visual characteristics of samples of cells treated with at least two agents;
calculating a Kolmogorov-Smirnov statistic for a particular agent, titration, and descriptor as compared to untreated control cells based on a continuous distribution function of the quantitated visual characteristic;
calculating z-scores by normalizing the Kolmogorov-Smirnov statistic for all descriptors and titrations based on the variability of the quantitated visual characteristic;
defining a titration sub-series by shifting the starting point of the titration series over a range of possible shifts;
calculating an s-correlation for each pair of titration sub-series for two agents; and
determining the value of s that yields the highest correlation between two titration subseries.

18. The method of claim 17, wherein agents are compared.

19. The method of claim 17, wherein descriptors are compared.

20. The method of claim 17 further comprising clustering of compounds or descriptors based on the s-correlation.
